(19) United States
(12) Patent Application Publication
(10) Pub. No.: US 2004/0175785 A1
(43) Pub. Date: Sep. 9, 2004

(54) Increasing bioavailability of carotenoids

(76) Inventors: Joseph Kanner, Rehovot (IL); Rina Granit, Rehovot (IL); Arieh Levy, Rehovot (IL)

Correspondence Address: Anthony Castorina, GE Ehrlich (1995) Ltd, Suite 207, 2001 Jefferson Davis Highway, Arlington, VA, 22202, US

(21) Appl. No.: 10/477,520

(22) PCT Filed: May 21, 2002
(86) PCT No.: PCT/IL02/00398

Related U.S. Application Data
(60) Provisional application No. 60/292,953, filed on May 24, 2001.

(30) Foreign Application Priority Data
Jul. 27, 2001 (US) 09/915,527

(51) Int. Cl. C12P 23/00

(52) U.S. Cl. 435/67

(57) ABSTRACT

A method of increasing a fraction of free carotenoids in a source of carotenoids in which at least some of the carotenoids are fatty acid esterified carotenoids is disclosed. The method is effected by contacting the source of carotenoids with an effective amount of an esterase under conditions effective in deesterifying the fatty acid esterified carotenoids, thereby increasing the fraction of free carotenoids in the source of carotenoids.

### FIELD AND BACKGROUND OF THE INVENTION

The present invention relates to a novel method of increasing the bioavailability of carotenoids. More particularly, the present invention relates to methods of extracting oleoresin, increasing the content of free carotenoids in sources of carotenoids rich in fatty acid esterified carotenoids, red pepper in particular. The present invention further relates to the extraction of free carotenoids from the sources of carotenoids rich in fatty acid esterified carotenoids and to food and feed additives that comprise free carotenoids.

Carotenoids, Chemistry and Biochemistry:

The carotenoids are isoprenoid compounds, with an extensive conjugated double bond system, and are biosynthesized from acetyl coenzyme-A via mevalonic acid as a branch of the great isoprenoid or terpenoid pathway (Britton, 1996). They are divided into two main classes; carotenes [acyclic (lycopene) and cyclic (β-carotene)], and xanthophylls (e.g., capsanthin). In contrast to carotenes, which are pure polyene hydrocarbons, xanthophylls also contain hydroxy, epoxy and keto groups. Only plants, and microorganisms synthesize carotenoids, however they are reach by feed and food animal or human tissues, which have the ability to absorb, modify and store these compounds (Goodwin; 1980).

Of the over 640 carotenoids found in nature, about 20 are present in a typical human diet. Of these carotenoids, only 14 and some of their metabolites have been identified in blood and tissues (Gerster, 1997; Khackick et al., 1995; Oshima, et al., 1997).

As part of the light-harvesting antenna, carotenoids can absorb photons and transfer the energy to chlorophyll, thus assisting in the harvesting of light in the range of 450-570 nm [see, Cogdell R J and Frank H A (1987) How carotenoids function in photosynthetic bacteria. Biochim Biophys Acta 895: 63-79; Cogdell R (1988) The function of pigments in chloroplasts. In: Goodwin T W (ed) Plant Pigments, pp 183-255. Academic Press, London; Frank H A, Violette C A, Trautman J K, Shreve A P, Owens T G and Albrecht A C (1991) Carotenoids in photosynthesis: structure and photochemistry. Pure Appl Chem 63: 109-114; Frank H A, Farhoosh R, Decoster B and Christensen R L (1992) Molecular features that control the efficiency of carotenoid-to-chlorophyll energy transfer in photosynthesis. In: Murata N (ed) Research in Photosynthesis, Vol I, pp 125-128. Kluwer, Dordrecht; and, Cogdell R J and Gardiner A T (1993) Functions of carotenoids in photosynthesis. Meth Enzymol 214: 185-193]. Although carotenoids are integral constituents of the protein-pigment complexes of the light-harvesting antennae in photosynthetic organisms, they are also important components of the photosynthetic reaction centers.

Most of the total carotenoids is located in the light harvesting complex II [Bassi R, Pineaw B, Dainese P and Marquartt J (1993) Carotenoid binding proteins of photosystem II. Eur J Biochem 212: 297-302]. The identities of the photosynthetically active carotenoproteins and their precise location in light-harvesting systems are not known. Carotenoids in photochemically active chlorophyll-protein complexes of the thermophilic cyanobacterium Synechococcus sp. were investigated by linear dichroism spectroscopy of oriented samples [see, Breton J and Kato S (1987) Orientation of the pigments in photosystem II: low-temperature linear-dichroism study of a core particle and of its chlorophyl]-protein subunits isolated from Synechococcus sp. Biochim Biophys Acta 892: 99-107]. These complexes contained mainly a β-carotene pool absorbing around 505 and 470 nm, which is oriented close to the membrane plane. In photochemically inactive chlorophyll-protein complexes, the β-carotene absorbs around 495 and 465 nm, and the molecules are oriented perpendicular to the membrane plane.

Evidence that carotenoids are associated with cyanobacterial photosystem (PS) II has been described [see, Suzuki R and Fujita Y (1977) Carotenoid photobleaching induced by the action of photosynthetic reaction center II: DCMU sensitivity. Plant Cell Physiol 18: 625-631; and, Newman P J and Sherman L A (1978) Isolation and characterization of photosystem I and II membrane particles from the blue-green alga Synechococcus cedrorum. Biochim Biophys Acta 503: 343-361]. There are two β-carotene molecules in the reaction center core of PS II [see, Ohno T, Satoh K and Katoh S (1986) Chemical composition of purified oxygen-evolving complexes from the thermophilic cyanobacterium Synechococcus sp. Biochim Biophys Acta 852: 1-8; Gounaris K, Chapman D J and Barber J (1989) Isolation and characterization of a D1/D2/cytochrome b-559 complex from Synechocystis PCC6803. Biochim Biophys Acta 973: 296-301; and, Newell R W, van Amerongen H, Barber J and van Grondelle R (1993) Spectroscopic characterization of the reaction center of photosystem II using polarized light: Evidence for ζ-carotene excitors in PS II reaction centers. Biochim Biophys Acta 1057: 232-238] whose exact function(s) is still obscure [reviewed by Satoh K (1992) Structure and function of PS II reaction center. In: Murata N (ed) Research in Photosynthesis, Vol. II, pp. 3-12. Kluwer, Dordrecht]. It was demonstrated that these two coupled β-carotene molecules protect chlorophyll P680 from photodamage in isolated PS II reaction centers [see, De Las Rivas J, Telfer A and Barber J (1993) 2-coupled β-carotene molecules protect P680 from photodamage in isolated PS II reaction centers. Biochim. Biophys. Acta 1142: 155-164], and this may be related to the protection against degradation of the D1 subunit of PS II [see, Sandmann G (1993) Genes and enzymes involved in the desaturation reactions from phytoene to lycopene. (abstract), 10th International Symposium on Carotenoids, Trondheim C L 1-2]. The light-harvesting pigments of a highly purified, oxygen-evolving PS II complex of the thermophilic cyanobacterium Synechococcus sp. consists of 50 chlorophyll αand 7 β-carotene, but no xanthophyll, molecules [see, Ohno T, Satoh K and Katoh S (1986) Chemical composition of purified oxygen-evolving complexes from the thermophilic cyanobacterium Synechococcus sp. Biochim Biophys Acta 852: 1-8]. β-carotene was shown to play a role in the assembly of an active PS II in green algae [see, Humbeck K, Romer S and Senger H (1989) Evidence for the essential role of carotenoids in the assembly of an active PS II. Planta 179: 242-250].

Isolated complexes of PS I from Phormidium luridum, which contained 40 chlorophylls per P700, contained an average of 1.3 molecules of β-carotene [see, Thornber J P, Alberte R S, Hunter F A, Shiozawa J A and Kan K S (1976) The organization of chlorophyll in the plant photosynthetic unit. Brookhaven Symp Biology 28: 132-148]. In a preparation of PS I particles from Synechococcus sp. strain PCC 6301, which contained 130+5 molecules of antenna chlorophylls per P700, 16 molecules of carotenoids were detected [see, Lundell D J, Glazer A N, Melis A and Malkin R (1985) Characterization of a cyanobacterial photosystem I complex. J Biol Chem 260: 646-654]. A substantial content of β-carotene and the xanthophylls cryptoxanthin and isocryptoxanthin were detected in PS I pigment-protein complexes of the thermophilic cyanobacterium Synechococcus elongatus [see, Coufal J, Hladik J and Sofrova D (1989) The carotenoid content of photosystem 1 pigment-protein complexes of the cyanobacterium Synechococcus elongatus. Photosynthetica 23: 603-616]. A subunit protein-complex structure of PS I from the thermophilic cyanobacterium Synechococcus sp., which consisted of four polypeptides (of 62, 60, 14 and 10 kDa), contained approximately 10 β-carotene molecules per P700 [see, Takahashi Y, Hirota K and Katoh S (1985) Multiple forms of P700-chlorophyll α-protein complexes from Synechococcus sp.: the iron, quinone and carotenoid contents. Photosynth Res 6: 183-192]. This carotenoid is exclusively bound to the large polypeptides which carry the functional and antenna chlorophyll a. The fluorescence excitation spectrum of these complexes suggested that β-carotene serves as an efficient antenna for PS I.

As mentioned, an additional essential function of carotenoids is to protect against photooxidation processes in the photosynthetic apparatus that are caused by the excited triplet state of chlorophyll. Carotenoid molecules with π-electron conjugation of nine or more carbon-carbon double bonds can absorb triplet-state energy from chlorophyll and thus prevent the formation of harmful singlet-state oxygen radicals. In Synechococcus sp. the triplet state of carotenoids was monitored in closed PS II centers and its rise kinetics of approximately 25 nanoseconds is attributed to energy transfer from chlorophyll triplets in the antenna [see, Schlodder E and Brettel K (1988) Primary charge separation in closed photosystem II with a lifetime of 11 nanoseconds. Flash-absorption spectroscopy with oxygen-evolving photosystem II complexes from Synechococcus. Biochim Biophys Acta 933: 22-34]. It is conceivable that this process, that has a lower yield compared to the yield of radical-pair formation, plays a role in protecting chlorophyll from damage due to over-excitation.

The protective role of carotenoids in vivo has been elucidated through the use of bleaching herbicides such as norflurazon that inhibit carotenoid biosynthesis in all organisms performing oxygenic photosynthesis [reviewed by Sandmann G and Boger P (1989) Inhibition of carotenoid biosynthesis by herbicides. In: Boger P and Sandmann G (Eds.) Target Sites of Herbicide Action, pp 25-44. CRC Press, Boca Raton, Fla.]. Treatment with norflurazon in the light results in a decrease of both carotenoid and chlorophyll levels, while in the dark, chlorophyll levels are unaffected. Inhibition of photosynthetic efficiency in cells of Oscillatoria agardhii that were treated with the pyridinone herbicide, fluridone, was attributed to a decrease in the relative abundance of myxoxanthophyll, zeaxanthin and β-carotene, which in turn caused photooxidation of chlorophyll molecules [see, Canto de Loura I, Dubacq J P and Thomas J C (1987) The effects of nitrogen deficiency on pigments and lipids of cianobacteria. Plant Physiol 83: 838-843].

It has been demonstrated in plants that zeaxanthin is required to dissipate, in a nonradiative manner, the excess excitation energy of the antenna chlorophyll [see, Demmig-Adams B (1990) Carotenoids and photoprotection in plants: a role for the xanthophyll zeaxanthin. Biochim Biophys Acta 1020: 1-24; and, Demmig-Adams B and Adams W W III (1990) The carotenoid zeaxanthin and high-energy-state quenching of chlorophyll fluorescence. Photosynth Res 25: 187-197]. In algae and plants a light-induced deepoxidation of violaxanthin to yield zeaxanthin, is related to photoprotection processes [reviewed by Demmig-Adams B and Adams W W III (1992) Photoprotection and other responses of plants to high light stress. Ann Rev Plant Physiol Plant Mol Biol 43: 599-626]. The light-induced deepoxidation of violaxanthin and the reverse reaction that takes place in the dark, are known as the “xanthophyll cycle” [see, Demmig-Adams B and Adams W W III (1992) Photoprotection and other responses of plants to high light stress. Ann Rev Plant Physiol Plant Mol Biol 43: 599-626]. Cyanobacterial lichens, that do not contain any zeaxanthin and that probably are incapable of radiation energy dissipation, are sensitive to high light intensity; algal lichens that contain zeaxanthin are more resistant to high-light stress [see, Demmig-Adams B, Adams W W III, Green T G A, Czygan F C and Lange O L (1990) Differences in the susceptibility to light stress in two lichens forming a phycosymbiodeme, one partner possessing and one lacking the xanthophyll cycle. Oecologia 84: 451-456; Demmig-Adams B and Adams W W III (1993) The xanthophyll cycle, protein turnover, and the high light tolerance of sun-acclimated leaves. Plant Physiol 103: 1413-1420; and, Demmig-Adams B (1990) Carotenoids and photoprotection in plants: a role for the xanthophyll zeaxanthin. Biochim Biophys Acta 1020: 1-24]. In contrast to algae and plants, cyanobacteria do not have a xanthophyll cycle. However, they do contain ample quantities of zeaxanthin and other xanthophylls that can support photoprotection of chlorophyll.

Several other functions have been ascribed to carotenoids. The possibility that carotenoids protect against damaging species generated by near ultra-violet (UV) irradiation is suggested by results describing the accumulation of β-carotene in a UV-resistant mutant of the cyanobacterium Gloeocapsa alpicola [see, Buckley C E and Houghton J A (1976) A study of the effects of near UV radiation on the pigmentation of the blue-green alga Gloeocapsa alpicola. Arch Microbiol 107: 93-97]. This has been demonstrated more elegantly in Escherichia coli cells that produce carotenoids [see, Tuveson R W and Sandmann G (1993) Protection by cloned carotenoid genes expressed in Escherichia coli against phototoxic molecules activated by near-ultraviolet light. Meth Enzymol 214: 323-330]. Due to their ability to quench oxygen radical species, carotenoids are efficient anti-oxidants and thereby protect cells from oxidative damage. This function of carotenoids is important in virtually all organisms [see, Krinsky N I (1989) Antioxidant functions of carotenoids. Free Radical Biol Med 7: 617-635; and, Palozza P and Krinsky N I (1992) Antioxidant effects of carotenoids in vivo and in vitro—an overview. Meth Enzymol 213: 403-420]. Other cellular functions could be affected by carotenoids, even if indirectly.

In flowers and fruits carotenoids facilitate the attraction of pollinators and dispersal of seeds. This latter aspect is strongly associated with agriculture. The type and degree of pigmentation in fruits and flowers are among the most important traits of many crops. This is mainly since the colors of these products often determine their appeal to the consumers and thus can increase their market worth.

Carotenoids have important commercial uses as coloring agents in the food industry since they are non-toxic [see, Bauemfeind J C (1981) Carotenoids as colorants and vitamin A precursors. Academic Press, London]. The red color of the tomato fruit is provided by lycopene which accumulates during fruit ripening in chromoplasts. Tomato extracts, which contain high content (over 80% dry weight) of lycopene, are commercially produced worldwide for industrial use as food colorant. Furthermore, the flesh, feathers or eggs of fish and birds assume the color of the dietary carotenoid provided, and thus carotenoids are frequently used in dietary additives for poultry and in aquaculture. Certain cyanobacterial species, for example Spirulina sp. [see, Sommer T R, Potts W T and Morrissy N M (1990) Recent progress in processed microalgae in aquaculture. Hydrobiologia 204/205: 435-443], are cultivated in aquaculture for the production of animal and human food supplements. Consequtntly, the content of carotenoids, primarily of β-carotene, in these cyanobacteria has a major commercial implication in biotechnology.

Most carotenoids are composed of a C₄₋₀ hydrocarbon backbone, constructed from eight C₅ isoprenoid units and contain a series of conjugated double bonds. Carotenes do not contain oxygen atoms and are either linear or cyclized molecules containing one or two end rings. Xanthophylls are oxygenated derivatives of carotenes. Various glycosilated carotenoids and carotenoid esters have been identified. The C₄₀ backbone can be further extended to give C₄₅ or C₅₀ carotenoids, or shortened yielding apocarotenoids. Some nonphotosynthetic bacteria also synthesize C₃₀ carotenoids. General background on carotenoids can be found in Goodwin T W (1980) The Biochemistry of the Carotenoids, Vol. 1, 2nd Ed. Chapman and Hall, New York; and in Goodwin T W and Britton G (1988) Distribution and analysis of carotenoids. In: Goodwin T W (ed) Plant Pigments, pp 62-132. Academic Press, New York.

More than 640 different naturally-occurring carotenoids have been so far characterized, hence, carotenoids are responsible for most of the various shades of yellow, orange and red found in microorganisms, fungi, algae, plants and animals. Carotenoids are synthesized by all photosynthetic organisms as well as several nonphotosynthetic bacteria and fungi, however they are also widely distributed through feeding throughout the animal kingdom.

Carotenoids are synthesized de novo from isoprenoid precursors only in photosynthetic organisms and some microorganisms, they typically accumulate in protein complexes in the photosynthetic membrane, in the cell membrane and in the cell wall.

In the biosynthesis pathway of β-carotene, four enzymes convert geranylgeranyl pyrophosphate of the central isoprenoid pathway to β-carotene. Carotenoids are produced from the general isoprenoid biosynthetic pathway. While this pathway has been known for several decades, only recently, and mainly through the use of genetics and molecular biology, have some of the molecular mechanisms involved in carotenoids biogenesis, been elucidated. This is due to the fact that most of the enzymes which take part in the conversion of phytoene to carotenes and xanthophylls are labile, membrane-associated proteins that lose activity upon solubilization [see, Beyer P, Weiss G and Kleinig H (1985) Solubilization and reconstitution of the membrane-bound carotenogenic enzymes from daffodile chromoplasts. Eur J Biochem 153: 341-346; and, Bramley P M (1985) The in vitro biosynthesis of carotenoids. Adv Lipid Res 21: 243-279].

Carotenoids are synthesized from isoprenoid precursors. The central pathway of isoprenoid biosynthesis may be viewed as beginning with the conversion of acetyl-CoA to mevalonic acid. D³-isopentenyl pyrophosphate (IPP), a C₅ molecule, is formed from mevalonate and is the building block for all long-chain isoprenoids. Following isomerization of IPP to dimethylallyl pyrophosphate (DMAPP), three additional molecules of IPP are combined to yield the C₂₀ molecule, geranylgeranyl pyrophosphate (GGPP). These 1′-4 condensation reactions are catalyzed by prenyl transferases [see, Kleinig H (1989) The role of plastids in isoprenoid biosynthesis. Ann Rev Plant Physiol Plant Mol Biol 40: 39-59]. There is evidence in plants that the same enzyme, GGPP synthase, carries out all the reactions from DMAPP to GGPP [see, Dogbo O and Camara B (1987) Purification of isopentenyl pyrophosphate isomerase and geranylgeranyl pyrophosphate synthase from Capsicum chromoplasts by affinity chromatography. Biochim Biophys Acta 920: 140-148; and, Laferriere A and Beyer P (1991) Purification of geranylgeranyl diphosphate synthase from Sinapis alba etioplasts. Biochim Biophys Acta 216: 156-163].

The first step that is specific for carotenoid biosynthesis is the head-to-head condensation of two molecules of GGPP to produce prephytoene pyrophosphate (PPPP). Following removal of the pyrophosphate, GGPP is converted to 15-cis-phytoene, a colorless C₄₋₀ hydrocarbon molecule. This tvo-step reaction is catalyzed by the soluble enzyme, phytoene synthase, an enzyme encoded by a single gene (crtB), in both cyanobacteria and plants [see, Chamovitz D, Misawa N, Sandmann G and Hirschberg J (1992) Molecular cloning and expression in Escherichia coli of a cyanobacterial gene coding for phytoene-synthase, a carotenoid biosynthesis enzyme. FEBS Lett 296: 305-310; Ray J A, Bird C R, Maunders M, Grierson D and Schuch W (1987) Sequence of pTOM5, a ripening related cDNA from tomato. Nucl Acids Res 15: 10587-10588; Camara B (1993) Plant phytoene synthase complex—component 3 enzymes, immunology, and biogenesis. Meth Enzymol 214: 352-365]. All the subsequent steps in the pathway occur in membranes. Four desaturation (dehydrogenation) reactions convert phytoene to lycopene via phytofluene, ζ-carotene, and neurosporene. Each desaturation increases the number of conjugated double bonds by two such that the number of conjugated double bonds increases from three in phytoene to eleven in lycopene.

Relatively little is known about the molecular mechanism of the enzymatic dehydrogenation of phytoene [see, Jones B L and Porter J W (1986) Biosynthesis of carotenes in higher plants. CRC Crit Rev Plant Sci 3: 295-324; and, Beyer P, Mayer M and Kleinig H (1989) Molecular oxygen and the state of geometric iosomerism of intermediates are essential in the carotene desaturation and cyclization reactions in daffodil chromoplasts. Eur J Biochem 184: 141-150]. It has been established that in cyanobacteria, algae and plants the first two desaturations, from 15-cis-phytoene to ζ-carotene, are catalyzed by a single membrane-bound enzyme, phytoene desaturase [see, Jones B L and Porter J W (1986) Biosynthesis of carotenes in higher plants. CRC Crit Rev Plant Sci 3: 295-324; and, Beyer P, Mayer M and Kleinig H (1989) Molecular oxygen and the state of geometric iosomerism of intermediates are essential in the carotene desaturation and cyclization reactions in daffodil chromoplasts. Eur J Biochem 184: 141-150]. Since the ζ-carotene product is mostly in the all-trans configuration, a cis-trans isomerization is presumed at this desaturation step. The primary structure of the phytoene desaturase polypeptide in cyanobacteria is conserved (over 65% identical residues) with that of algae and plants [see, Pecker I, Chamovitz D, Linden H, Sandmann G and Hirschberg J (1992) A single polypeptide catalyzing the conversion of phytoene to ζ-carotene is transcriptionally regulated during tomato fruit ripening. Proc Natl Acad Sci USA 89: 4962-4966; Pecker I, Chamovitz D, Mann V, Sandmann G, Boger P and Hirschberg J (1993) Molecular characterization of carotenoid biosynthesis in plants: the phytoene desaturase gene in tomato. In: Murata N (ed) Research in Photosynthesis, Vol III, pp 11-18. Kluwer, Dordrectht]. Moreover, the same inhibitors block phytoene desaturase in the two systems [see, Sandmann G and Boger P (1989) Inhibition of carotenoid biosynthesis by herbicides. In: Boger P and Sandmann G (eds) Target Sites of Herbicide Action, pp 25-44. CRC Press, Boca Raton, Fla.]. Consequently, it is very likely that the enzymes catalyzing the desaturation of phytoene and phytofluene in cyanobacteria and plants have similar biochemical and molecular properties, that are distinct from those of phytoene desaturases in other microorganisms. One such a difference is that phytoene desaturases from Rhodobacter capsulatus, Erwinia sp. or fungi convert phytoene to neurosporene, lycopene, or 3,4-dehydrolycopene, respectively.

Desaturation of phytoene in daffodil chromoplasts [see, Beyer P, Mayer M and Kleinig H (1989) Molecular oxygen and the state of geometric iosomerism of intermediates are essential in the carotene desaturation and cyclization reactions in daffodil chromoplasts. Eur J Biochem 184: 141-150], as well as in a cell free system of Synechococcus sp. strain PCC 7942 [see, Sandmann G and Kowalczyk S (1989) In vitro carotenogenesis and characterization of the phytoene desaturase reaction in Anacystis. Biochem Biophys Res Com 163: 916-921], is dependent on molecular oxygen as a possible final electron acceptor, although oxygen is not directly involved in this reaction. A mechanism of dehydrogenase-electron transferase was supported in cyanobacteria over dehydrogenation mechanism of dehydrogenase-monooxygenase [see, Sandmann G and Kowalczyk S (1989) In vitro carotenogenesis and characterization of the phytoene desaturase reaction in Anacystis. Biochem Biophys Res Com 163: 916-921]. A conserved FAD-binding motif exists in all phytoene desaturases whose primary structures have been analyzed [see, Pecker I, Chamovitz D, Linden H, Sandmann G and Hirschberg J (1992) A single polypeptide catalyzing the conversion of phytoene to ζ-carotene is transcriptionally regulated during tomato fruit ripening. Proc Natl Acad Sci USA 89: 4962-4966; Pecker I, Chamovitz D, Mann V, Sandmann G, Boger P and Hirschberg J (1993) Molecular characterization of carotenoid biosynthesis in plants: the phytoene desaturase gene in tomato. In: Murata N (ed) Research in Photosynthesis, Vol III, pp 11-18. Kluwer, Dordrectht]. The phytoene desaturase enzyme in pepper was shown to contain a protein-bound FAD [see, Hugueney P, Romer S, Kuntz M and Camara B (1992) Characterization and molecular cloning of a flavoprotein catalyzing the synthesis of phytofluene and ζ-carotene in Capsicum chromoplasts. Eur J Biochem 209: 399-407]. Since phytoene desaturase is located in the membrane, an additional, soluble redox component is predicted. This hypothetical component could employ NAD(P)⁺, as suggested [see, Mayer M P, Nievelstein V and Beyer P (1992) Purification and characterization of a NADPH dependent oxidoreductase from chromoplasts of Narcissus pseudonarcissus—a redox-mediator possibly involved in carotene desaturation. Plant Physiol Biochem 30: 389-398] or another electron and hydrogen carrier, such as a quinone. The cellular location of phytoene desaturase in Synechocystis sp. strain PCC 6714 and Anabaena variabilis strain ATCC 29413 was determined with specific antibodies to be mainly (85%) in the photosynthetic thylakoid membranes [see, Serrano A, Gimenez P, Schmidt A and Sandmann G (1990) Immunocytochemical localization and functional determination of phytoene desaturase in photoautotrophic prokaryotes. J Gen Microbiol 136: 2465-2469].

In cyanobacteria algae and plants ζ-carotene is converted to lycopene via neurosporene. Very little is known about the enzymatic mechanism, which is predicted to be carried out by a single enzyme [see, Linden H, Vioque A and Sandmann G (1993) Isolation of a carotenoid biosynthesis gene coding for ζ-carotene desaturase from Anabaena PCC 7120 by heterologous complementation. FEMS Microbiol Lett 106: 99-104]. The deduced amino acid sequence of ζ-carotene desaturase in Anabaena sp. strain PCC 7120 contains a dinucleotide-binding motif that is similar to the one found in phytoene desaturase.

Two cyclization reactions convert lycopene to ζ-carotene. Evidence has been obtained that in Synechococcus sp. strain PCC 7942 [see, Cunningham F X Jr, Chamovitz D, Misawa N, Gantt E and Hirschberg J (1993) Cloning and functional expression in Escherichia coli of a cyanobacterial gene for lycopene cyclase, the enzyme that catalyzes the biosynthesis of β-carotene. FEBS Lett 328: 130-138], as well as in plants [see, Camara B and Dogbo 0 (1986) Demonstration and solubilization of lycopene cyclase from Capsicum chromoplast membranes. Plant Physiol 80: 172-184], these two cyclizations are catalyzed by a single enzyme, lycopene cyclase. This membrane-bound enzyme is inhibited by the triethylamine compounds, CPTA and MPTA [see, Sandmann G and Boger P (1989) Inhibition of carotenoid biosynthesis by herbicides. In: Boger P and Sandmann G (eds) Target Sites of Herbicide Action, pp 25-44. CRC Press, Boca Raton, Fla.]. Cyanobacteria carry out only the 1-cyclization and therefore do not contain ε-carotene, δ-carotene and α-carotene and their oxygenated derivatives. The β-ring is formed through the formation of a “carbonium ion” intermediate when the C-1,2 double bond at the end of the linear lycopene molecule is folded into the position of the C-5,6 double bond, followed by a loss of a proton from C-6. No cyclic carotene has been reported in which the 7,8 bond is not a double bond. Therefore, full desaturation as in lycopene, or desaturation of at least half-molecule as in neurosporene, is essential for the reaction. Cyclization of lycopene involves a dehydrogenation reaction that does not require oxygen. The cofactor for this reaction is unknown. A dinucleotide-binding domain was found in the lycopene cyclase polypeptide of Synechococcus sp. strain PCC 7942, implicating NAD(P) or FAD as coenzymes with lycopene cyclase.

The addition of various oxygen-containing side groups, such as hydroxy-, methoxy-, oxo-, epoxy-, aldehyde or carboxylic acid moieties, form the various xanthophyll species. Little is known about the formation of xanthophylls. Hydroxylation of β-carotene requires molecular oxygen in a mixed-function oxidase reaction.

Clusters of genes encoding the enzymes for the entire pathway have been cloned from the purple photosynthetic bacterium Rhodobacter capsulatus [see, Armstrong G A, Alberti M, Leach F and Hearst J E (1989) Nucleotide sequence, organization, and nature of the protein products of the carotenoid biosynthesis gene cluster of Rhodobacter capsulatus. Mol Gen Genet 216: 254-268] and from the nonphotosynthetic bacteria Erwinia herbicola [see, Sandmann G, Woods W S and Tuveson R W (1990) Identification of carotenoids in Erwinia herbicola and in transformed Escherichia coli strain. FEMS Microbiol Lett 71: 77-82; Hundle B S, Beyer P, Kleinig H, Englert H and Hearst J E (1991) Carotenoids of Erwinia herbicola and an Escherichia coli HB 101 strain carrying the Erwinia herbicola carotenoid gene cluster. Photochem Photobiol 54: 89-93; and, Schnurr G, Schmidt A and Sandmann G (1991) Mapping of a carotenogenic gene cluster from Erwinia herbicola and functional identification of six genes. FEMS Microbiol Lett 78: 157-162] and Erwinia uredovora [see, Misawa N, Nakagawa M, Kobayashi K, Yamano S, Izawa I, Nakamura K and Harashima K (1990) Elucidation of the Erwinia uredovora carotenoid biosynthetic pathway by functional analysis of gene products in Escherichia coli. J Bacteriol 172: 6704-6712]. Two genes, al-3 for GGPP synthase [see, Nelson M A, Morelli G, Carattoli A, Romano N and Macino G (1989) Molecular cloning of a Neurospora crassa carotenoid biosynthetic gene (albino-3) regulated by blue light and the products of the white collar genes. Mol Cell Biol 9: 1271-1276; and, Carattoli A, Romano N, Ballario P, Morelli G and Macino G (1991) The Neurospora crassa carotenoid biosynthetic gene (albino 3). J Biol Chem 266: 5854-5859] and al-1 for phytoene desaturase [see, Schmidhauser T J, Lauter F R, Russo V EA and Yanofsky C (1990) Cloning sequencing and photoregulation of al-1, a carotenoid biosynthetic gene of Neurospora crassa. Mol Cell Biol 10: 5064-5070] have been cloned from the fungus Neurospora crassa. However, attempts at using these genes as heterologous molecular probes to clone the corresponding genes from cyanobacteria or plants were unsuccessful due to lack of sufficient sequence similarity.

The first “plant-type” genes for carotenoid synthesis enzyme were cloned from cyanobacteria using a molecular-genetics approach. In the first step towards cloning the gene for phytoene desaturase, a number of mutants that are resistant to the phytoene-desaturase-specific inhibitor, norflurazon, were isolated in Synechococcus sp. strain PCC 7942 [see, Linden H, Sandmann G, Chamovitz D, Hirschberg J and Boger P (1990) Biochemical characterization of Synechococcus mutants selected against the bleaching herbicide norflurazon. Pestic Biochem Physiol 36: 46-51]. The gene conferring norflurazon-resistance was then cloned by transforming the wild-type strain to herbicide resistance [see, Chamovitz D, Pecker I and Hirschberg J (1991) The molecular basis of resistance to the herbicide norflurazon. Plant Mol Biol 16: 967-974; Chamovitz D, Pecker I, Sandmann G, Boger P and Hirschberg J (1990) Cloning a gene for norflurazon resistance in cyanobacteria. Z Naturforsch 45c: 482-486]. Several lines of evidence indicated that the cloned gene, formerly called pds and now named crtP, codes for phytoene desaturase. The most definitive one was the functional expression of phytoene desaturase activity in transformed Escherichia coli cells [see, Linden H, Misawa N. Chamovitz D, Pecker I, Hirschberg J and Sandmann G (1991) Functional complementation in Escherichia coli of different phytoene desaturase genes and analysis of accumulated carotenes. Z Naturforsch 46c: 1045-1051; and, Pecker I, Chamovitz D, Linden H, Sandmann G and Hirschberg J (1992) A single polypeptide catalyzing the conversion of phytoene to ζ-carotene is transcriptionally regulated during tomato fruit ripening. Proc Natl Acad Sci USA 89: 4962-4966]. The crtP gene was also cloned from Synechocystis sp. strain PCC 6803 by similar methods [see, Martinez-Ferez I M and Vioque A (1992) Nucleotide sequence of the phytoene desaturase gene from Synechocystis sp. PCC 6803 and characterization of a new mutation which confers resistance to the herbicide norflurazon. Plant Mol Biol 18: 981-983].

The cyanobacterial crtP gene was subsequently used as a molecular probe for cloning the homologous gene from an alga [see, Pecker I, Chamovitz D, Mann V, Sandmann G, Boger P and Hirschberg J (1993) Molecular characterization of carotenoid biosynthesis in plants: the phytoene desaturase gene in tomato. In: Murata N (ed) Research in Photosynthesis, Vol III, pp 11-18. Kluwer, Dordrectht] and higher plants [see, Bartley G E, Viitanen P V, Pecker I, Chamovitz D, Hirschberg J and Scolnik P A (1991) Molecular cloning and expression in photosynthetic bacteria of a soybean cDNA coding for phytoene desaturase, an enzyme of the carotenoid biosynthesis pathway. Proc Natl Acad Sci USA 88: 6532-6536; and, Pecker I, Chamovitz D, Linden H, Sandmann G and Hirschberg J (1992) A single polypeptide catalyzing the conversion of phytoene to ζ-carotene is transcriptionally regulated during tomato fruit ripening. Proc Natl Acad Sci USA 89: 4962-4966]. The phytoene desaturases in Synechococcus sp. strain PCC 7942 and Synechocystis sp. strain PCC 6803 consist of 474 and 467 amino aeid residues, respectively, whose sequences are highly conserved (74% identities and 86% similarities). The calculated molecular mass is 51 kDa and, although it is slightly hydrophobic (hydropathy index −0.2), it does not include a hydrophobic region which is long enough to span a lipid bilayer membrane. The primary structure of the cyanobacterial phytoene desaturase is highly conserved with the enzyme from the green alga Dunalliela bardawil (61% identical and 81% similar; [see, Pecker I, Chamovitz D, Mann V, Sandmann G, Boger P and Hirschberg J (1993) Molecular characterization of carotenoid biosynthesis in plants: the phytoene desaturase gene in tomato. In: Murata N (ed) Research in Photosynthesis, Vol III, pp 11-18. Kluwer, Dordrectht]) and from tomato [see, Pecker I, Chamovitz D, Linden H, Sandmann G and Hirschberg J (1992) A single polypeptide catalyzing the conversion of phytoene to ζ-carotene is transcriptionally regulated during tomato fruit ripening. Proc Natl Acad Sci USA 89: 4962-4966], pepper [see, Hugueney P, Romer S, Kuntz M and Camara B (1992) Characterization and molecular cloning of a flavoprotein catalyzing the synthesis of phytofluene and ζ-carotene in Capsicum chromoplasts. Eur J Biochem 209: 399-407] and soybean [see, Bartley G E, Viitanen P V, Pecker I, Chamovitz D, Hirschberg J and Scolnik P A (1991) Molecular cloning and expression in photosynthetic bacteria of a soybean cDNA coding for phytoene desaturase, an enzyme of the carotenoid biosynthesis pathway. Proc Natl Acad Sci USA 88: 6532-6536] (62-65% identical and 79% similar; [see, Chamovitz D (1993) Molecular analysis of the early steps of carotenoid biosynthesis in cyanobacteria: Phytoene synthase and phytoene desaturase. Ph.D. Thesis, The Hebrew University of Jerusalem]). The eukaryotic phytoene desaturase polypeptides are larger (64 kDa); however, they are processed during import into the plastids to mature forms whose sizes are comparable to those of the cyanobacterial enzymes.

There is a high degree of structural similarity in carotenoid enzymes of Rhodobacter capsulatus, Erwinia sp. and Neurospora crassa [reviewed in Armstrong G A, Hundle B S and Hearst J E (1993) Evolutionary conservation and structural similarities of carotenoid biosynthesis gene products from photosynthetic and nonphotosynthetic organisms. Meth Enzymol 214: 297-311], including in the crtI gene-product, phytoene desaturase. As indicated above, a high degree of conservation of the primary structure of phytoene desaturases also exists among oxygenic photosynthetic organisms. However, there is little sequence similarity, except for the FAD binding sequences at the amino termini, between the “plant-type” crtP gene products and the “bacterial-type” phytoene desaturases (crtI gene products; 1.9-23% identities and 42-47% similarities). It has been hypothesized that crtP and crtI are not derived from the same ancestral gene and that they originated independently through convergent evolution [see, Pecker I, Chamovitz D, Linden H, Sandmann G and Hirschberg J (1992) A single polypeptide catalyzing the conversion of phytoene to ζ-carotene is transcriptionally regulated during tomato fruit ripening. Proc Natl Acad Sci USA 89: 4962-4966]. This hypothesis is supported by the different dehydrogenation sequences that are catalyzed by the two types of enzymes and by their different sensitivities to inhibitors.

Although not as definite as in the case of phytoene desaturase, a similar distinction between cyanobacteria and plants on the one hand and other microorganisms is also seen in the structure of phytoene synthase. The crtB gene (formerly psy) encoding phytoene synthase was identified in the genome of Synechococcus sp. strain PCC 7942 adjacent to crtP and within the same operon [see, Bartley G E, Viitanen P V, Pecker I, Chamovitz D, Hirschberg J and Scolnik P A (1991) Molecular cloning and expression in photosynthetic bacteria of a soybean cDNA coding for phytoene desaturase, an enzyme of the carotenoid biosynthesis pathway. Proc Natl Acad Sci USA 88: 6532-6536]. This gene encodes a 36-kDa polypeptide of 307 amino acids with a hydrophobic index of −0.4. The deduced amino acid sequence of the cyanobacterial phytoene synthase is highly conserved with the tomato phytoene synthase (57% identical and 70% similar; Ray J A, Bird C R, Maunders M, Grierson D and Schuch W (1987) Sequence of pTOM5, a ripening related cDNA from tomato. Nucl Acids Res 15: 10587-10588]) but is less highly conserved with the crtB sequences from other bacteria (29-32% identical and 48-50% similar with ten gaps in the alignment). Both types of enzymes contain two conserved sequence motifs also found in prenyl transferases from diverse organisms [see, Bartley G E, Viitanen P V, Pecker I, Chamovitz D, Hirschberg J and Scolnik P A (1991) Molecular cloning and expression in photosynthetic bacteria of a soybean cDNA coding for phytoene desaturase, an enzyme of the carotenoid biosynthesis pathway. Proc Natl Acad Sci USA 88: 6532-6536; Carattoli A, Romano N, Ballario P, Morelli G and Macino G (1991) The Neurospora crassa carotenoid biosynthetic gene (albino 3). J Biol Chem 266: 5854-5859; Armstrong G A, Hundle B S and Hearst J E (1993) Evolutionary conservation and structural similarities of carotenoid biosynthesis gene products from photosynthetic and nonphotosynthetic organisms. Meth Enzymol 214: 297-311; Math S K, Hearst J E and Poulter C D (1992) The crtE gene in Erwinia herbicola encodes geranylgeranyl diphosphate synthase. Proc Natl Acad Sci USA 89: 6761-6764; and, Chamovitz D (1993) Molecular analysis of the early steps of carotenoid biosynthesis in cyanobacteria: Phytoene synthase and phytoene desaturase. Ph.D. Thesis, The Hebrew University of IS Jerusalem]. It is conceivable that these regions in the polypeptide are involved in the binding and/or removal of the pyrophosphate during the condensation of two GGPP molecules.

The crtQ gene encoding ζ-carotene desaturase (formerly zds) was cloned from Anabaena sp. strain PCC 7120 by screening an expression library of cyanobacterial genomic DNA in cells of Escherichia coli carrying the Erwinia sp. crtB and crtE genes and the cyanobacterial crtP gene [see, Linden H, Vioque A and Sandmann G (1993) Isolation of a carotenoid biosynthesis gene coding for ζ-carotene desatura-se from Anabaena PCC 7120 by heterologous complementation. FEMS Microbiol Lett 106: 99-104]. Since these Escherichia coli cells produce ζ-carotene, brownish-red pigmented colonies that produced lycopene could be identified on the yellowish background of cells producing ζ-carotene. The predicted ζ-carotene desaturase from Anabaena sp. strain PCC 7120 is a 56-kDa polypeptide which consists of 499 amino acid residues. Surprisingly, its primary structure is not conserved with the “plant-type” (crtP gene product) phytoene desaturases, but it has considerable sequence similarity to the bacterial-type enzyme (crtI gene product) [see, Sandmann G (1993) Genes and enzymes involved in the desaturation reactions from phytoene to lycopene. (abstract), 10th International Symposium on Carotenoids, Trondheim CL1-2]. It is possible that the cyanobacterial crtQ gene and crtI gene of other microorganisms originated in evolution from a common ancestor.

The crtL gene for lycopene cyclase (formerly icy) was cloned from Synechococcus sp. strain PCC 7942 utilizing essentially the same cloning strategy as for crtP. By using an inhibitor of lycopene cyclase, 2-(4-methylphenoxy)-triethylamine hydrochloride (MPTA), the gene was isolated by transformation of the wild-type to herbicide-resistance [see, Cunningham F X Jr, Chamovitz D, Misawa N, Gantt E and Hirschberg J (1993) Cloning and functional expression in Escherichia coli of a cyanobacterial gene for lycopene cyclase, the enzyme that catalyzes the biosynthesis of β-carotene. FEBS Lett 328: 130-138]. Lycopene cyclase is the product of a single gene product and catalyzes the double cyclization reaction of lycopene to β-carotene. The crtL gene product in Synechococcus sp. strain PCC 7942 is a 46-kDa polypeptide of 411 amino acid residues. It has no sequence similarity to the crtY gene product (lycopene cyclase) from Erwinia uredovora or Erwinia herbicola.

The gene for β-carotene hydroxylase (criZ) and zeaxanthin glycosilase (crtX) have been cloned from Erwinia herbicola [see, Hundle B, Alberti M, Nievelstein V, Beyer P, Kleinig H, Armstrong G A, Burke D H and Hearst J E (1994) Functional assignment of Ervinia herbicola Eho10 carotenoid genes expressed in Escherichia coli. Mol Gen Genet 254: 406-416; Hundle B S, Obrien D A, Alberti M, Beyer P and Hearst J E (1992) Functional expression of zeaxanthin glucosyltransferase from Erwinia herbicola and a proposed diphosphate binding site. Proc Natl Acad Sci USA 89: 9321-9325] and from Erwinia uredovora [see, Misawa N, Nakagawa M, Kobayashi K, Yamano S, Izawa 1, Nakamura K and Harashima K (1990) Elucidation of the Erwinia uredovora carotenoid biosynthetic pathway by functional analysis of gene products in Escherichia coli. J Bacteriol 172: 6704-6712].

Caroteizoids as Anitioxidants:

Most carotenoids are efficient antioxidants, quenching singlet oxygen (102) and scavenging peroxyl radicals (Sies and Stahl, 1995). 102, 02-, H₂O₂ and peroxyl radicals are reactive oxygen species generated in biological cells. All these species may react with DNA, proteins and lipids impairing their physiological functions (Halliwell, 1996). Such processes are discussed as initial events in the pathogenesis of several diseases including cancer, cardiovascular diseases, or age-related system degeneration. Carotenoids inactivate singlet oxygen via physical or chemical quenching. The efficacy of physical quenching exceeds that of chemical quenching by far, 99.9%, and involves that transfer of excitation energy from ¹O₂ to the carotenoid. In the process of physical quenching the carotenoid remains intact, so that it can undergo further cycles of singlet oxygen quenching. Methylene blue was used as a sensitizer to study the consumption of carotenoids during photooxidation of human plasma and LDL (Ojima et al., 1993). Lycopene, β-carotene and xanthophylls were found to decrease photooxidation in blood plasma while they remained unchanged (Wagner et al., 1993). Hirayama et al (−1994) investigated the singlet oxygen quenching ability of 18 carotenoids and reported that the xanthophylls conjugated keto group enhanced quenching, while hydroxy, epoxy and methoxy groups showed lesser effects.

Capsanthin and capsorubin were found to act as better singlet oxygen quenchers than β-carotene. Previous studies show that β-carotene is a good scavenger of hypochlorite and others have demonstrated its scavenging ability of nitrogen dioxide. (Kanner et al., 1983, Everett et al., 1996).

Carotenoids are efficient scavengers of peroxyl radicals, especially at low oxygen tension (Burton and Ingold, 1984; Kennedy and Liebler, 1992). The interaction of carotenoids with peroxyl radicals generated by the azo compounds AMVN and AAPH in a phosphatidylcholine liposome system were investigated by Lin et al (1992). In this system the xanthophylls astaxanthin, zeaxanthin and cantaxanthin were more efficient free radical scavengers than ζ-carotene. However, investigating the reaction of carotenoids with peroxyl free radical in emulsion showed that lycopene and β-carotene are better scavengers than several xanthophylls (Woodall et al., 1997). Matsufuji et al. (1998) investigated the radical scavenging ability of carotenoids in methyl linoleate emulsion and demonstrated that capsanthin acts better than lutein, zeaxanthin and β-carotene.

Oxidative modification of low-density lipoproteins (LDL), which is thought to be a key step in early atherosclerosis, is protected by the lipoprotein-associated antioxidants. LDL contains about 1 carotenoid and 12 α-tocopherol molecules per LDL particle, a relatively small number compared with about 2,300 molecules of oxidizable lipid in each LDL particle (Romanchik et al., 1995). Some antioxidant supplements, such as α-tocopherol consistently appear to enhance the ability of LDL to resist oxidation, (Esterbauer et al., 1991; Aviram, 1999). However, α-carotene shows less consistent protective ability (Gaziano et al., 1995; Reaven et al., 1994). In contrast, Lin et al. (1998) showed that depletion of β-carotene in healthy women increased the susceptibility of LDL to oxidation, whereas a normal intake provide protection to LDL. Most recently, dietary supplementation with β-carotene, but not lycopene was shown to inhibit endothelial cell—mediated ex-vivo per oxidation of LDL (Dugas et al., 1999). Mixture of carotenoids have been found to be more effective than any single carotenoid in protecting liposomes against lipid peroxidation (Stahl et al., 1998), and as antioxidants in membranes and LDL. Moreover, it has been reported that carotenoids enhance vitamin E antioxidant efficiency (Bohm et al., 1997; Fuhrman et al., 1997; Fuhrman and Aviram, 1999).

Atherosclerosis and LDL Oxidation as Affected by Carotenoids During Atherogenesis:

Atherosclerosis is the major cause of morbidity and mortality in the western world and its pathogenesis involves complicated interacting among cells of the arterial wall, blood cells, and plasma lipoproteins (Ross, 1993). Macrophage cholesterol accumulation and foam cell formation are the indications of early atherogenesis with most of the cholesterol in these cells derived from plasma low-density lipoproteins (LDL). The most studied modification of LDL with a potential pathological significance is LDL oxidation (Steinberg et al., 1989). The involvement of oxidized LDL in atherosclerosis is suggested from its presence in the atherosclerotic lesion in human and of the apolipoprotein E deficient (E⁰) mice (Yla-Herttula et al., 1989; Aviram et al., 1995), from the increased susceptibility to oxidation of LDL derived from atherosclerotic patients and also from the anti-atherogenecity of several dietary antioxidants (Steinberg et al., 1992; Frankel et al., 1993; Aviram, 1996).

High-density lipoproteins (HDL) are associated with anti-atherogenic activity and HDL levels are inversely related to the risk of developing atherosclerosis. Paraoxonase, an enzyme, physically associated in serum with HDL, has been shown to be inversely related to the risks of atherogenesis (Watson et al., 1995; Aviram, 1999). The LDL oxidation hypothesis of atherosclerosis raised an extensive investigation into the role of antioxidants against LDL oxidation as a possible preventive treatment for atherosclerosis. Efforts are made to identif natural food products, which offer antioxidant defense against LDL oxidation.

Consumption of flavonoids in the diet has been shown to be inversely associated with morbidity from coronary heat disease, (Hertog et al., 1993; Knekt et al., 1996). Flavonoids extracted from red wine protected LDL oxidation where added in-vitro (Frankel et al., 1993) and consumption of red wine was shown to inhibit LDL oxidation ex-vivo (Kondo, 1994; Fuhrman et al., 1995).

Carotenoid consumption has been shown in previous epidemiological studies to be associated with reduced cardiovascular mortality (Kohlmeier and Hasting, 1995). However, several dietary intervention trials involving β-carotene have yielded inconclusive results (Mayne, 1996). Lee et al. (1999) reported that among healthy women given a β-carotene supplement for a limited time, no benefit or harm was observed regarding incidence of cancer and of cardiovascular diseases. Lower serum lycopene levels were associated with increase risk and mortality from coronary heart disease in a cross sectional study of Lithuanian and Swedish populations (Kristenson et al., 1997; Rao and Agarwal, 1999). Iribarren et al. (1997) found the xanthophylls lutein and zeaxanthin to be the carotenoid with the strongest inverse association with extreme carotid artery intima-medial thickening.

Cancer and the Effects of Carotenoids:

Cancer development is characterized by specific cellular transformations followed by uncontrolled cell growth and invasion of the tumor site with a potential for subsequent detachment, transfer into the blood stream and metastases formation at distal site(s) (Ilyas et al., 1999). All these stages involve a number of cellular alterations including changes in proliferation rates, inactivation of tumor suppressor genes and inhibition of apoptosis (Goldsworthy et al., 1996; Knudsen-et al., 1999; Ilyas et al., 1999).

Dietary exposures provide one of the environmental factors believed to be significant in the etiology of a number of epithe/loid cancer cases, notably oral and colon carcinomas. Cancer inhibitory properties for a number of micronutrients with antioxidant properties have been demonstrated in recent years mainly in experimental animal models (Jain et al., 1999), in cell culture studies (Schwartz and Shklar, 1992), and in some human studies (Schwartz et al., 1991). Epidemiological evidence links nutrition rich in vegetables and fruits, with reduced risks of degenerative disease, the evidence is particular compelling for cancer (Block et al., 1992). Epidemiological studies suggest that the incidence of human cancer is inversely correlated with the dietary intake of carotenoids and their concentration in plasma (Ziegler, 1988). A variety of carotenoids are present in commonly eaten foods and these compounds accumulate in tissues and blood plasma. Animal studies and cultured cell studies have shown that many carotenoids such as α-carotene, β-cryptoxanthin, astaxanthin and lycopene have anticarcinogenic activities. (Murakoshi et al., 1992; Tanaka et al., 1995; Levy et al., 1995). However, there have been contradictory reports concerning the use of β-carotene for cancer prevention (Hannekens et al., 1996). A multicenter case-control study to evaluate the relation between antioxidant status and cancer has shown that lycopene but not β-carotene, contribute to the protective effect of vegetable consumption (Kohlmeier et al., 1997).

The putative biological mechanisms of cancer inhibition of the antioxidant micronutrients are:

(1) Enhancement of production of cytotoxic immune cells and production of cytokines (Schwartz et al., 1990).

(2) Activation of cancer suppressor genes such as wild p53 (Schwartz et al., 1993), or deactivation of oncogenes such as Ha-ras and mutated p53 (Schwartz et al., 1992).

(3) Inhibition of angiogenesis-stimulating factors involved with tumor angiogenesis (Schwartz and Shklar, 1997).

Primary prevention or drug-based therapeutics of oral and colon cancer is a public health goal but still not feasible despite major advances in understanding of the mechanisms at the genetic, germline, somatic, immunologic and angiogenic levels. Therefore, a great interest in preventive nutrition has arisen focusing on the role of dietary components with antioxidant activity such as several vitamins and carotenoids, to prevent cancer (Weisburger, 1999).

Oral Cancer:

The frequency of oral cancer is 4-5% of all cancer cases in the western world. Squamous cell carcinoma (SCC) make up 95.% of oral cancer cases. Risk factors in oral cancer include tobacco as a major risk factor, and alcohol abuse, especially when used in combination with tobacco (De Stefani et al., 1998; Hart et al., 1999; Schildt et al., 1998; Dammer et al., 1998; Bundgaard et al., 1995). Viral Infections, particularly with several species of Human Papilloma Virus (HPV) have been associated with both benign and malignant oral lesions (Smith et al., 1998).

Leukoplakia is the most common pre-neoplastic condition. Leukoplakia presents as white lesions on the oral mucosa, while erythroleukoplakia is a variant of leukoplakia in which the clinical presentation includes erythematous area as well. When biopsied, leukoplakia may show a spectrum of histologic changes ranging from hyperkeratosis, dysplasia to carcinoma-in-situ or even invasive carcinoma. Dysplastic changes are more frequent in erythroleokoplakia. Leukoplakia is considered a pre-neoplastic lesion, which carries a 15% risk for malignant transformation over time if dysplasia is not diagnosed in the initial biopsy, and up to 36% transformation for lesions with dysplasia at the time of first biopsy (Mao, 1997). Leukoplakia is associated with the use of tobacco in the majority of cases, but cases of leukoplakia in non-smoking women, have a higher risk. When leukoplakia is diagnosed, the treatment protocol consists of cessation of risk habits, and frequent follow-up, including repeated biopsies. No effective long-term preventive treatment is yet available.

Ki67, PCNA, CyclinD1, p53, p16, and p21 are all cell cycle associated proteins, which are over-expressed in oral cancer and pre-cancer, and are associated with a negative prognosis in cancer cases (Schoelch et al., 1999; Yao et al., 1999; Birchall et al., 1999).

The Role of Carotenoids in the Prevention of Oral Cancer:

Vitamin A and its derivatives, by way of systemic administration or topical application have been shown to be beneficial in regressing leukoplakia. In cases of oral cancer, vitamin-A and its derivatives have been shown to reduce the risk of secondary cancer (Hong et al., 1990; Gravis et al., 1999). However, in long term use they are associated with significant side effects, and the lesions tend to recur when treatment is discontinued. Beta-carotenes are not associated with significant side effects, and there is evidence from experimental studies that indicate they may be effective in inhibiting malignant transformation, however, there is contradictory data regarding their efficiency in clinical use for oral cancer and pre-cancer (Stich et al., 1998). A recent study has shown significantly lower levels of serum ζ-carotene and of tissue ζ-carotene in smokers, which are at risk for developing oral cancer (Cowan et al., 1999).

The prognosis of oral cancer is generally poor. The mean five-year survival of oral cancer cases is only about 50%, and although much improved diagnostic and treatment tools have been introduced, survival has not improved over the last two decades.

Treatment consists of surgery radiation and chemotherapy, and in most cases is associated with severe effects on the quality of life, such as impaired esthetics, mastication, and speech.

In view of the poor prognosis of oral cancer, prevention and regression at the pre-malignant stage is of enormous importance. However when a pre-malignant lesion such as leukoplakia is identified, very few efficient treatment modalities are yet available for routine practice. Therefore, a continuing effort is necessary to identify new compounds that will be able to regress existing lesions and prevent their transformation into malignancy, with minimal or no side effects, to allow for long term use in patients at risk. It is also important to find chemopreventing agents that will reduce the risk for secondary cancer in patients with primary oral cancer, which is as high as 36%.

Colon Cancer:

Colon cancer is the third most common form of cancer and the overall estimated new cases per year worldwide represent about 10% of all new cancer cases. The disease is most frequent in Occidental countries including Israel. Epidemiological studies have emphasized the major role of diet in the ethiology of colon cancer. Attempts to identify causative or protective factors in epidemiological and experimental studies have led to some discrepancies. Nonetheless, prospects for colorectal cancer control are bright and a number of possible approaches could prove fruitful. Bras and associates (1999) have recently demonstrated that in familial adenomatous polyposis patients, a population highly prone to develop colorectal cancer, exhibit an imbalance in the pro-oxidant/antioxidant status. In addition, the levels of ascorbate and tocopherol were considerably lower in this population. Collins et al. (1998) have shown in populations from five different European countries that the mean 8-oxodeoxyguanosine (8-oxo-dG) concentrations in lymphocyte DNA showed a significant positive correlation with colorectal cancer. It would appear that patients with colonic cancer undergo a significant reduction in their antioxidant reserve compared to healthy subjects. These studies support the notion that one approach to identify protective factors in colorectal canter will be those that provide a balanced oxidative status, or fit the antioxidant hypothesis. This hypothesis proposes that vitamin C, vitamin E, and carotenoids occurring in fruits and vegetables afford protection against cancer by preventing oxidative damage to lipids and to DNA.

The Role of Carotenoids in the Prevention of Colon Cancer:

Recent studies suggest a protective effect of carotenoids and antioxidants, lycopene and lycopene-rich tomatoes against various cancers, among them, colon cancer.

Rats with induced colon cancer fed lycopene or tomato juice/water solution, had shown a lower colon cancer incidence than the control group. The protective effect against colon preneoplasia associated with enhanced antioxidant properties was observed in a study where rats were administered a carcinogen and administered lycopene in the form of 6% oleoresin supplementation (Jain et al., 1999). Chemoprevention by lycopene of mouse lung neoplasia has also been reported (Kim et al., 1997). Kim et al. (1988) assessed the effect of carotenoids, such as fucoxanthin, lutein and phenolics such curcumin and its derivative tetrahydrocurcumin (THC) on colon cancer development in mice. Flucoxanthin, lutein, carcumin and THC significantly decreased the number of aberrant crypt foci compared to the control group. Proliferation rate was lower following this treatment, with higher effectiveness seen by THC. A similar effect was reported by Narisawa and associates (1996) with the exception for β-carotene.

Human studies conducted by Pappalardo et al., (1997), compared the status of carotenoids in tissue and plasma in healthy subjects and subjects with pre-cancer and cancerous lesions. The cancer subjects had lower levels of carotenoid than those of healthy subjects.

Genetic and Breeding of Red Pepper:

Red pepper is one of the richest sources of carotenoids among vegetable crops. Most of the domesticated varieties of red pepper belong to the species Capsicum annuum; pepper breeding has focused and evolved mainly on the development of cultivars and varieties suited for use as a vegetable, spice condiment, ornamental or medicinal plant. Few studies have been devoted to the improvement of the chemical and nutritional composition of peppers (Bosland, 1993; Poulos, 1994). Capsanthin is the predominant carotenoid of the red pepper fruit and its content is controlled by major genes and polygenes; several genes have been identified along its biosynthetic pathway (Lefebvre, 1998).

Carotenoids From Red Pepper Fruits:

Red pepper fruits, especially from paprika cultivars are used in the form of powders and oleoresins as food colorants. These products are very rich in carotenoids, some of them specific to pepper fruits. The keto carotenoid, capsanthin, occur only in red pepper, represents 50% the carotenoids in the vegetable and contribute to the red color. Zeaxanthin and lutein, β-carotene and β-cryptoxanthin are the additional carotenoids found in red pepper at concentrations of 20%, 10% and 5%, respectively (Levy et al., 1995). Capsanthin accounts for 30-60% of total carotenoids in fully ripe fruits. The capsanthin is esterified with fatty acids (nonesterified 20%; monoesterified 20-30%; diesterified 40-50%). The fatty acids of esterified capsanthins are chiefly lauric (12:0), myristic (14:0) and palmitic (16:0) acid.

Increasing the carotenoid concentration in high-pigment fruits of red pepper by genetic manipulation seems to improve not only the quality of the fruit as a food colorani but also as an important source of carotenoids, particularly, capsanthin. It was found that the breeding line 4126 contains about 240 mg carotenoids/100 grams fresh weight of which 120 mg are capsanthin (Levy et al., 1995). Tomatoes contain about 5 mg lycopene/100 grams fresh weight, and only in special breeding lines, levels of 15 mg lycopene/100 grams fresh weight are achieved. These enormous differences in carotenoid content emphasizes the high potential of red pepper cultivars as an appropriate food source with high carotenoid concentration.

Bioavailability of Carotenoids:

As a result of their lipophilic nature, carotenoids are often found complexed in the food matrix with proteins, lipids and or fiber. Several steps are necessary for carotenoid absorption to occur. The food matrix must be digested and the carotenoids must be released, physically and biochemically, and combined with lipids and bile salts to form micelles. The micelles must move to the intestinal brush border membrane for absorption and be transported into the enterocyte for subsequent processing. The chylomicrons move to the liver and are transported by lipoproteins for distribution to the different organs. Part of the carotenoids in chylomicrons remnants are taken up by extra-hepatic tissues before hepatic uptake (Lee et al., 1999). Thus, many factors influence absorption and hence bioavailability of dietary carotenoids. Humans absorb a variety of carotenoids intact, and some carotenoids such, as β-carotene, β-cryptoxanthin and α-carotene can contribute to the vitamin A status of the individual (Olson, 1999). Mathews-Roth et al. (1990) studied the absorption and distribution of (¹⁴C) canthaxanthin, a typical xanthophyll, and (14C) lycopene, an acyclic hydrocarbon carotenoid, in rats and rhesus monkeys. They showed that the liver accumulated the largest amount of both, however clearance of lycopene was much slower than canthaxanthin. Stahl and Sies (1992) showed that the lycopene concentration in human plasma was increased by the consumption of heat-processed tomato juice. Recently it was found in humans that in a single ingestion of paprika juice containing 34.2 μmole capsanthin and a week later tomato soup, containing 186.3 pmole lycopene, resulted in elevation of plasma carotenoids from both sources. The plasma contain only deesterified carotenoids including non-esterified capsanthin. The results also show that capsanthin disappear from the plasma more rapidly than lycopene (Oshima et al., 1997). Rainbow trout were fed diet supplemented with canthaxanthin and oleoresin paprika. Canthaxanthin was more efficient absorbed in the flesh of rainbow trout than paprika carotenoids (Akhtar et al., 1999).

Bioavailability of Carotenoids Esterified with Fatty Acids:

The bioavailability of paprika carotenoids in human and animal were shown to be lower than β-carotene or canthaxanthin (Akhtar et al., 1999). One of the reason to this reduced absorption seems to occur because most of the carotenoids are in an ester form with fatty acids. It is shown herein that pancreatic lipase catalyze the deesterification of paprika carotenoids to a very limited extent. This could explain the low bioavailability of carotenoids from paprika in animals.

Thus although the red pepper fruit is the richest in carotenoids of all other sources, the bioavailability of red pepper carotenoids is poor because red pepper carotenoids are esterified with fatty acids, which prevent their efficient uptake in the gut.

There is thus a widely recognized need for, and it would be highly advantageous to have, a method of deesterification of esterified carotenoids, so as to render such carotenoids bioavailable to human and animal.

### SUMMARY OF THE INVENTION

According to one aspect of the present invention there is provided a method of extracting red pepper oleoresin, the method comprising homogenizing red-pepper fruits in water into a juice; centrifuging the juice so as to obtain a pellet; mixing the pellet with ethanol and ethyl acetate; homogenizing the pellet with the ethanol and the ethyl acetate; removing dry material; and evaporating solvents so as to obtain red pepper oleoresin.

According to further features in preferred embodiments of the invention described below, a weight ratio between the red-pepper fruits and the water is 80-120 parts of fruit to 20-60 parts of water.

According to still further features in the described preferred embodiments the red-pepper fruits are frozen.

According to still further features in the described preferred embodiments the red-pepper fruits are fresh.

According to still further features in the described preferred embodiments the juice is centrifuged at 20,000-30,000 g for 10-30 minutes.

According to still further features in the described preferred embodiments the pellet is mixed with 1-3 parts of the ethanol and 5-15 parts of the ethyl acetate.

According to still further features in the described preferred embodiments removing the dry material is by centrifugation.

According to still further features in the described preferred embodiments evaporating the solvents is at 40-50° C.

According to still further features in the described preferred embodiments evaporating the solvents is under vacuum.

According to another aspect of the present invention there is provided a method of determining an efficiency of an esterase in increasing a fraction of free carotenoids in a source of carotenoids in which at least some of the carotenoids are fatty acid esterified carotenoids, the method comprising contacting the source of carotenoids with the esterase under preselected experimental conditions; and using a carotenoids detection assay for determining the efficiency of the esterase in increasing the fraction of the free carotenoids in the source of carotenoids.

According to still another aspect of the present invention there is provided a method of screening for esterases efficient in increasing a fraction of free carotenoids in a source of carotenoids in which at least some of the carotenoids are fatty acid esterified carotenoids, the method comprising contacting the source of carotenoids separately with each of the esterases under preselected experimental conditions; and using a carotenoids detection assay for determining the efficiency of each of the esterases in increasing the fraction of the free carotenoids in the source of carotenoids, thereby screening for esterases efficient in increasing the fraction of free carotenoids in the source of carotenoids.

According to yet another aspect of the present invention there is provided a method of optimizing reaction conditions for increasing a fraction of free carotenoids in a source of carotenoids in which at least some of the carotenoids are fatty acid esterified carotenoids, via an esterase, the method comprising contacting the source of carotenoids with the esterase under different preselected experimental conditions; and using a carotenoids detection assay for determining the efficiency of the esterase in increasing the fraction of the free carotenoids in the source of carotenoids under each of the different preselected experimental conditions, thereby optimizing the reaction conditions for increasing the fraction of free carotenoids in the source of carotenoids in which at least some of the carotenoids are fatty acid esterified carotenoids via the esterase.

According to still another aspect of the present invention there is provided a method of increasing a fraction of free carotenoids in a source of carotenoids in which at least some of the carotenoids are fatty acid esterified carotenoids, the method comprising contacting the source of carotenoids with an effective amount of an esterase under conditions effective in deesterifying the fatty acid esterified carotenoids, thereby increasing the fraction of free carotenoids in the source of carotenoids.

According to further features in preferred embodiments of the invention described below, the method further comprising extracting free carotenoids from the source of carotenoids.

According to an additional aspect of the present invention there is provided a source of carotenoids having an increased fraction of free carotenoids and produced by the method described herein.

According to an additional aspect of the present invention there is provided a food additive comprising the source of carotenoids having an increased fraction of free carotenoids as described herein.

According to an additional aspect of the present invention there is provided a feed additive comprising the source of carotenoids having an increased fraction of free carotenoids as described herein.

According to further features in preferred embodiments of the invention described below, the source of carotenoids is characterized in that a majority of the carotenoids in the source of carotenoids are the fatty acid esterified carotenoids.

According to still further features in the described preferred embodiments the source of carotenoids is red pepper.

According to still further features in the described preferred embodiments the source of carotenoids is red pepper powder.

According to still further features in the described preferred embodiments the source of carotenoids is paprika.

According to still further features in the described preferred embodiments the source of carotenoids is red pepper oil extract.

According to still further features in the described preferred embodiments the source of carotenoids is red pepper oleoresin.

According to still further features in the described preferred embodiments the source of carotenoids is selected from the group consisting of apple, apricot, avocado, blood orange cape gooseberry, carambola, chilli, clementine, kumquat, loquat, mango, minneola, nectarine, orange, papaya, peach, persimmon, plum, potato, pumpkin, tangerine and zucchini. According to still further features in the described preferred embodiments the esterase is selected from the group consisting of a lipase, a carboxyl ester esterase and a chlorophylase, preferably a lipase.

According to still further features in the described preferred embodiments the lipase is selected from the group consisting of bacterial lipase, yeast lipase, mold lipase and animal lipase.

According to still further features in the described preferred embodiments the esterase is immobilized.

According to still further features in the described preferred embodiments the preselected experimental conditions, the different preselected experimental conditions and/or the conditions effective in deesterifying the fatty acid esterified carotenoids, comprise at least one of addition of a cellulose degrading enzyme; addition of a pectin degrading enzyme; addition of an emulsifier; and addition of at least one metal ion.

According to still further features in the described preferred embodiments the at least one metal ion is selected from the group consisting of Ca++ and Na⁺.

According to still further features in the described preferred embodiments the addition of the at least one metal ion is by addition of at least one salt of said metal ion.

According to still further features in the described preferred embodiments the at least one salt is selected from the group consisting of CaCl₂ and NaCl.

According to still further features in the described preferred embodiments the cellulose degrading enzyme is selected from the group consisting of C 1 type beta-1,4 glucanase, exo-beta-1,4 glucanase, endo-beta-1,4 glucanase and beta-glucosidase.

According to still further features in the described preferred embodiments the proteins degrading enzyme is selected from the group consisting of tripsin, papain, chymotripsins, ficin, bromelin, cathepsins and rennin.

According to still further features in the described preferred embodiments the pectin degrading enzyme is selected from the group consisting of a pectinestrerase, pectate lyase and a polygalacturonase.

According to still further features in the described preferred embodiments the emulsifier is a non-ester emulsifier.

According to still further features in the described preferred embodiments the emulsifier is lecithin.

According to still further features in the described preferred embodiments the emulsifier is deoxycholate.

According to still further features in the described preferred embodiments the emulsifier is a non-ionic detergent, such as, but not limited to, polyoxyethylensorbitane monolaurate (TWEEN-20).

According to still further features in the described preferred embodiments the emulsifier is derived from bile, gum—Arabic or sodium salt of free fatty acids.

According to still further features in the described preferred embodiments the carotenoids detection assay is a chromatography assay.

According to still further features in the described preferred embodiments the chromatography assay is selected from the group consisting of thin layer chromatography and high performance liquid chromatography.

The present invention successfully addresses the shortcomings of the presently known configurations by providing methods of determining an efficiency of an esterase in increasing a fraction of free carotenoids in a source of carotenoids in which at least some of the carotenoids are fatty acid esterified carotenoids; screening for esterases efficient in increasing a fraction of free carotenoids in a source of carotenoids in which at least some of the carotenoids are fatty acid esterified carotenoids; optimizing reaction conditions for increasing a fraction of free carotenoids in a source of carotenoids in which at least some of the carotenoids are fatty acid esterified carotenoids, via an esterase; and increasing a fraction of free carotenoids in a source of carotenoids in which at least some of the carotenoids are fatty acid esterified carotenoids; and a source of carotenoids having an increased fraction of free carotenoids, which can serve as a food and/or feed additive; and a rich source from which one can extract to purification desired carotenoids.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice

In the drawings:

FIG. 1 is a HPLC chromatogram of natural red pepper carotenoids (obtained from oleoresin).

FIG. 2 is a HPLC chromatogram of natural red pepper (paprika) carotenoids following chemical saponification, the chromatogram contains mostly about 9 peaks of: (i) capsanthin (6.1 min); (ii) violaxanthin (7.36 min); (iii) capsanthin (8.89 min); (iv) cis-capsanthin (10.33); (v) capsolutein (10.83 min); (vi) Zeaxanthin (11.2 min); (vii) cis-Zeaxanthin (12.0 min); (viii) β-crypotxanthin (14.36 min); and (ix) β-carotene.

FIG. 3 is a HPLC chromatogram of natural red pepper (paprika) carotenoids following treatment with pectinase, protease, cellulase and lipase in the presence of deoxycholate.

FIG. 4 is a HPLC chromatogram of paprika oleoresin carotenoids following treatment with deoxycholate and lipase.

FIGS. 5a-c are HPLC chromatograms of paprika oleoresin carotenoids following treatment with varying concentarations of deoxycholate (2%, 3% and 4%, respectively) and lipase.

FIG. 6 demonstrates the steps of a method of extracting oleoresin from red pepper fruits, according to the present invention.

### DESCRIPTION OF THE PREFERRED EMBODIMENTS

The present invention is of methods of (i) determining an efficiency of an esterase in increasing a fraction of free carotenoids in a source of carotenoids in which at least some of the carotenoids are fatty acid esterified carotenoids; (ii) screening for esterases efficient in increasing a fraction of free carotenoids in a source of carotenoids in which at least some of the carotenoids are fatty acid esterified carotenoids; (iii) optimizing reaction conditions for increasing a fraction of free carotenoids in a source of carotenoids in which at least some of the carotenoids are fatty acid esterified carotenoids, via an esterase; (iv) increasing a fraction of free carotenoids in a source of carotenoids in which at least some of the carotenoids are fatty acid esterified carotenoids; and (iv) an efficient method of extracting red pepper oleoresin. The present invention is further of a source of carotenoids having an increased fraction of free carotenoids, which can serve as a food and/or feed additive and as a rich source from which to extract to substantial purification desired carotenoids.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

According to one aspect of the present invention there is provided a method of extracting red pepper oleoresin. The red pepper fruit can be either fresh or frozen. The method is effected homogenizing red-pepper fruits in water into a juice; centrifuging the juice so as to obtain a pellet; mixing the pellet (either directly or after freezing) with ethanol and ethyl acetate; homogenizing the pellet with the ethanol and the ethyl acetate; removing dry material; and evaporating solvents so as to obtain red pepper oleoresin.

As is further detailed and exemplified hereinbelow, esterified carotenoids can be deesterified from the pellet (directly or after freezing), or, preferably, from the oleoresin derived therefrom via extraction as descried above, by a lipase preferably in the presence of a cellulase and a pectinase.

Preferably, a weight ratio between the red-pepper fruits and the water is 80-120 parts of fruit to 20-60 parts of water. Still preferably, the juice is centrifuged at 20,000-30,000 g for 10-30 minutes. Yet preferably, the pellet is mixed with 1-3 parts of the ethanol and 5-15 parts of the ethyl acetate. Still preferably, removing the dry material is by centrifugation. Preferably, evaporating the solvents is at 40-50° C. and preferably under vacuum.

According to another aspect of the present invention there is provided a method of determining an efficiency of an esterase in increasing a fraction of free carotenoids in a source of carotenoids in which at least some of the carotenoids are fatty acid esterified carotenoids. The method according to this aspect of the present invention is effected by contacting the source of carotenoids with the esterase under preselected experimental conditions; and using a carotenoids detection assay for determining the efficiency of the esterase in increasing the fraction of the free carotenoids in the source of carotenoids.

According to still another aspect of the present invention there is provided a method of screening for esterases efficient in increasing a fraction of free carotenoids in a source of carotenoids in which at least some of the carotenoids are fatty acid esterified carotenoids. The method according to this aspect of the present invention is effected by contacting the source of carotenoids separately with each of the esterases under preselected experimental conditions; and using a carotenoids detection assay for determining the efficiency of each of the esterases in increasing the fraction of the free carotenoids in the source of carotenoids, thereby screening for esterases efficient in increasing the fraction of free carotenoids in the source of carotenoids.

According to yet another aspect of the present invention there is provided a method of optimizing reaction conditions for increasing a fraction of free carotenoids in a source of carotenoids in which at least some of the carotenoids are fatty acid esterified carotenoids, via an esterase. The method according to this aspect of the present invention is effected by contacting the source of carotenoids with the esterase under different preselected experimental conditions; and using a carotenoids detection assay for determining the efficiency of the esterase in increasing the fraction of the free carotenoids in the source of carotenoids under each of the different preselected experimental conditions, thereby optimizing the reaction conditions for increasing the fraction of free carotenoids in the source of carotenoids in which at least some of the carotenoids are fatty acid esterified carotenoids via the esterase.

Preferably, the carotenoids detection assay is a chromatography assay, such as, but not limited to, thin layer chromatography (TLC) and high performance liquid chromatography (HPLC). These assays are well known for, and are frequently used in the characterization of different carotenoids.

According to still another aspect of the present invention there is provided a method of increasing a fraction of free carotenoids in a source of carotenoids in which at least some of the carotenoids are fatty acid esterified carotenoids. The method according to this aspect of the present invention is effected by contacting the source of carotenoids with an effective amount of an esterase under conditions effective in deesterifying the fatty acid esterified carotenoids, thereby increasing the fraction of free carotenoids in the source of carotenoids. Once freed, individual non-esterified carotenoids or groups of similar non-esterified carotenoids can be extracted and purified to substantial homogeneity using methods well known in the art, such as, but not limited to, extraction with organic solvents followed by phase separation, various chromatographies, etc.

The source of carotenoids, rich in free, non-esterified carotenoids, produced by the method of the present invention, and/or the free carotenoids further purified therefrom can be used as food and/or feed additives in human or animal diet, to serve as natural antioxidants and/or food, animal and cosmetic natural colorants.

A preferred source of carotenoids according to the present invention is characterized in that a majority of the carotenoids in the source of carotenoids are fatty acid esterified carotenoids, such as, for example, red pepper derived carotenoids. Red pepper is one of the richest sources of carotenoids among vegetable crops. Most of the domesticated varieties of red pepper belong to the species Capsicum annuum; pepper breeding has focused and evolved mainly on the development of cultivars and varieties suited for use as a vegetable, spice condiment, ornamental or medicinal plant. Few studies have been devoted to the improvement of the chemical and nutritional composition of peppers (Bosland, 1993; Poulos, 1994). Capsanthin is the predominant carotenoid of the red pepper fruit and its content is controlled by major genes and polygenes; several genes have been identified along its biosynthetic pathway (Lefebvre, 1998).

Red pepper fruits, especially from paprika cultivars are used in the form of powders and oleoresins as food colorants. These products are very rich in carotenoids, some of them specific to pepper fruits. The keto carotenoid, capsanthin, occur only in red pepper, represents 50% the carotenoids in the vegetable and contribute to the red color. Zeaxanthin and lutein, β-carotene and β-cryptoxanthin are the additional carotenoids found in red pepper at concentrations of 20%, 10% and 5%, respectively (Levy et al., 1995). Capsanthin accounts for 30-60% of total carotenoids in fully ripe fruits. The capsanthin is esterified with fatty acids (nonesterified 20%; monoesterified 20-30%; diesterified 40-50%). The fatty acids of esterified capsanthins are chiefly lauric (12:0), myristic (14:0) and palmitic (16:0) acid. The bioavailability of fatty acids esterified carotenoids is, nevertheless, very low.

Other plant species that containing fatty acid esterified carotenoids, including, but not limited to, apple, apricot, avocado, blood orange cape gooseberry, carambola, chilli, clementine, kumquat, loquat, mango, minneola, nectarine, orange, papaya, peach, persimmon, plum, potato, pumpkin, tangerine and zucchini, can also be used as a source of carotenoids for the present invention. The esterified carotenoids content of these fruits are described in Dietmar E. Breithaupt and Ameneh Bamedi “Carotenoid ester in vegetables and fruits: A screening with emphasis on beta-cryptoxanthin esters” J. Agric. Food Chem. 2001, 49, 2064-2070, which is incorporated herein by reference.

Any type of esterase that can deesterify fatty acid esterified carotenoids can be used to implement the present invention. Methods for screening for most efficient esterases and suitable conditions for their activity with respect to different types of substrates (carotenoids sources) are also described herein. The esterase of choice can be, for example, a lipase, a carboxyl ester esterase or a chlorophylase, preferably a lipase. Enzymes species belonging to these families are known to deesterify a wide range of fatty acid esters, i.e., to have a wide range of substrate specificity. Different lipases can be used in the method of the present invention, including, for example, those obtained from bacterial, yeast or animal sources. The esterase used while implementing the methods of the present invention can be free in solution or immobilized. In either case, as is further detailed below, an oil-in-water or preferably water-in-oil emulsion of the carotenoid source is prepared in order to enhance catalytic activity of the esterase employed. Other means to enhance enzyme activity can also be practiced, depending to a large extent on the source of carotenoids, such means are further discussed below.

Lipases typically catalyze the deesterification of triglycerides and diglycerides containing fatty acids bond to glycerol by ester bond. The carotenoids in, for example, paprika are esterified by fatty acids such as myristic, lauric, palmitic stearic, oleic and linoleic acids and for this reason they are different from triglycerides which are the natural substrates for lipases. Lipases are known to hydrolyze emulsified acyl lipids, as they are active on a water/lipid interface. For this reason, deoxycholate improves the reaction of the enzyme and its concentration is important to receive a high reactivity of the enzymes. Lipase catalyzed reactions are accelerated by Ca²⁺ ions since the freed fatty acids are precipitated as insoluble Ca-salts. Introduction of Ca²⁺ ions in the process described herein enhances deesterification.

Thus, according to preferred embodiments of the present invention, the preselected experimental conditions, the different preselected experimental conditions and/or the conditions effective in deesterifying the fatty acid esterified carotenoids, comprise, for example, the addition of a cellulose degrading enzyme; the addition of a proteins degrading enzyme; the addition of a pectin degrading enzyme; the addition of an emulsifier to the reaction mixture; and/or the addition of at least one metal ion to the reaction mixture, e.g., the addition of salts, such as CaCl2 and/or Nacl. Other reaction conditions such as the addition of effectors, temperature, pH, etc., can also be optimized for each combination of enzyme and substrate.

The degrading enzymes used in context of the present invention serve to degrade their respective substrates present in the reaction mixture in order to avoid sequestering effects and reduce the viscosity of the reaction mixture.

The cellulose of choice can be a C₁ type beta-1,4 glucanase, exo-beta-1,4 glucanase, endo-beta-1,4 glucanase and/or beta-glucosidase from plant, insect or bacterial source. The proteins degrading enzyme can be, for example, tripsin, papain, chymotripsins, ficin, bromelin, cathepsins and/or rennin. The type and amount of the proteins degrading enzyme is controlled so as to avoid degradation of the esterase itself. The pectin degrading enzyme can, for example, be a pectinestrerase, pectate lyase and/or a polygalacturonase.

Careful attention should be given to the emulsifier of choice. Lipid esterases are water soluble and therefore reside in the water component of the emulsion, yet, their substrates reside in the oily portion of the emulsion. Preferably, the emulsifier employed is a non-ester emulsifier, as ester emulsifiers can adversely affect the reaction as competitive substrates or inhibitors of the esterase of choice. Presently referred emulsifiers hence include lecithin, deoxycholate, gum Arabic (e.g., 0.5-2.0%), free fatty acid salts (e.g., 0.5-2.0%), bile derived emulsifiers and non-ionic detergents, such as, but not limited to, polyoxyethylensorbitane monolaurate (TWEEN-20).

The present invention provides methods of (i) determining an efficiency of an esterase in increasing a fraction of free carotenoids in a source of carotenoids in which at least some of the carotenoids are fatty acid esterified carotenoids; (ii) screening for esterases efficient in increasing a fraction of free carotenoids in a source of carotenoids in which at least some of the carotenoids are fatty acid esterified carotenoids; (iii) optimizing reaction conditions for increasing a fraction of free carotenoids in a source of carotenoids in which at least some of the carotenoids are fatty acid esterified carotenoids, via an esterase; and (iv) increasing a fraction of free carotenoids in a source of carotenoids in which at least some of the carotenoids are fatty acid esterified carotenoids. The present invention further provide a source of carotenoids having an increased fraction of free carotenoids, which can serve as a food and/or feed additive; and a rich source from which one can extract to purification desired carotenoids.

The present invention offers a great advantage over processes for chemical deesterification of carotenoids. For example, alkaline treatment of paprika affects to a great extent the properties of its proteins and antioxidants such as vitamin C and E. It will be appreciated that during heating of paprika to high temperatures, as required in alkaline based deesterification of carotenoids, one or more of the following adverse reactions takes place: (i) destruction of essential amino acids; (ii) conversion of natural amino acids into derivatives which are not metabolized; (iii) decrease of the digestibility of proteins as a result of cross-linking; and, last, but not least, generation of cytotoxic compounds. It will be appreciated in this respect that due to the formation, at high pH values, of enolates, phenolic compounds, including vitamin E and most of the other antioxidants are more rapidly oxidized, in a process that generates free radicals which oxidize and destroy carotenoids (Belitz and Grosch W. Food Chemistry, Springer-Verlag, 1987).

Additional objects, advantages, and novel features of the present invention will become apparent to one ordinarily skilled in the art upon 20 examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### EXAMPLE 1

### Materials and Experimental Procedures

Materials:

Paprika powder and oleoresin paprika were purchased from Tavlinei-Hanegev, Avshalom. Sodium phosphate, citric acid, TWEEN-20 (polyoxyethylensorbitane monolaurate) and potassium hydroxide were obtained from Merck (Darmstadt, Germany). Deoxycholic acid (sodium salt) BHT (Butylated hydroxy toluene), lipase pancreatic from porcine were obtained from Sigma Chemical Co. (St. Louis, Mo). The enzymes, lipase A “Amano 6”, lipase F-AP15 and lipase AY “Amano 30” (approved for human consumption) were from Amano, Pharmaceuticals Co. LTD (Nishiki, Japan). Pectinase/cellulase, Rohameut Max and protease (Coralase PN-L) were obtained from Rohm Enzyme gmbh (Darmstadt, Germany). HPLC grade ethanol and hexane were from Biolab (Israel) and HPLC acetone from Baker (Deventer, Holland).

High-Performance Liquid Chromatography (HPLC):

HPLC was conducted on a Shimadzu LC-10 AT equipped with SCL-10A Shimadzu diode array detector. Photodiode array measurements of spectral properties from the individual peaks (from 260 to 540 nm) were determined at the upslope, apex and downslope. The column (Merck RP-18e 3.4×250 mM, 5-μm particles) was used for HPLC separations. The peaks were detected at 450 and 474 nm. The mobile phase were acetone and H₂O with a gradient suggested by Minguez-Mosquera et al. 1993 (J. Agric. Food Chem. 41, 1616-1620).

Deesterification Paprika Powder by Enzymes:

Paprika powder (500 mg) was suspended in 9.5 ml water in the presence of Cellulase-Pectinase (100 μl), Lipase (100 mg) and 0.2% deoxycholate (200 mg) at pH 4.93. The suspension was Shaken in a heated bath at 37° C. for 24 hours. Carotenoids were extracted from these suspension by addition of ethanol (5 ml) and 5 ml of hexane. The extraction with hexane was done repeatedly until no color could be observed in the extracts.

Deesteriflcation paprika oleoresin by enzymes: Paprika oleoresin (20 mg) was mixed with TWEEN-20 (200 μl) or deoxycholate (100 mg) and 10 ml of H₂O. The emulsion has been shaken at 37° C. for 24 hours. Extraction of carotenoids was performed by the addition of 4 ml of ethanol and 5 ml of hexane. The extraction with hexane was done repeatedly until no color could be observed in the extracts. The combined hexane extracts were washed with water (25 ml) and dried over anhydrous sodium sulfate for HPLC determination of the carotenoids.

Chemical Deesterification (Chemical Saponification):

Chemical deesterification was performed essentially as described in Ittah et al., J. Agric. Food Chem. 1993, 41, 899-901.

### EXAMPLE 2

### Experimental Results

FIG. 1 demonstrates a chromatogram of natural red pepper (paprika) carotenoids. The main carotenoid is capsanthin. The free unesterified capsanthin was eluted at about 9 min. Most of the capsanthin is esterified as monoesters and diesters. The mono esters were eluted in three major peaks after β-cryptoxanthin (14.33 min) and before β-carotene (18.9 min). The diesters were eluted as 7 major peaks between 22-26 min.

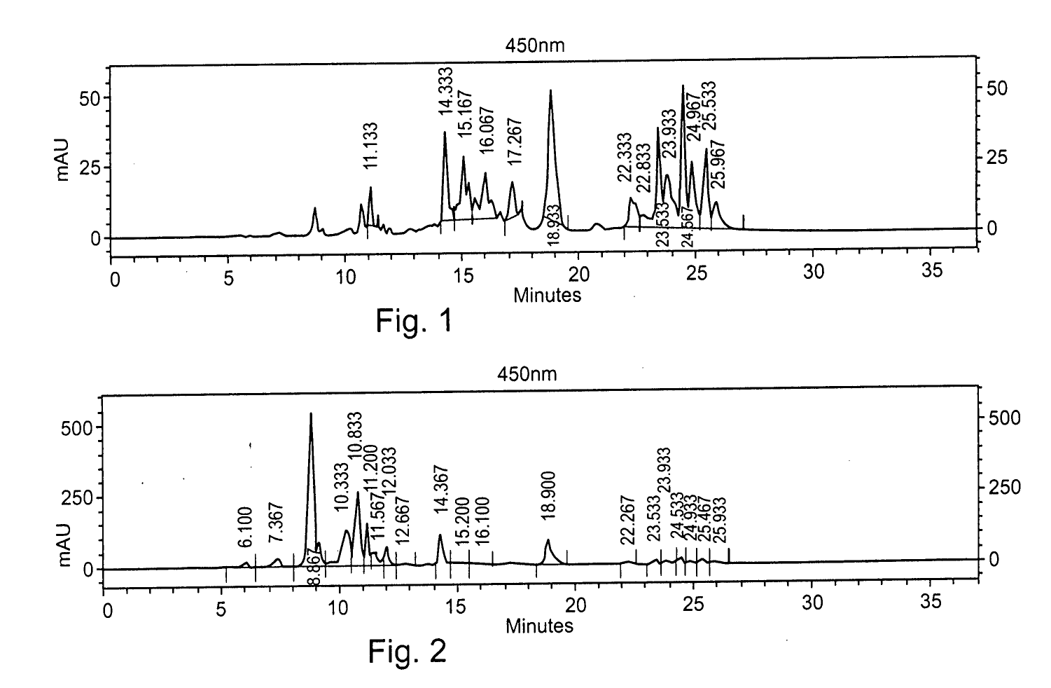

FIG. 2 demonstrates that following chemical saponification all the peaks of red pepper (paprika) diesters and monoesters carotenoids disappeared and the chromatogram contains mostly about 9 peaks of: (i) capsanthin (6.1 min); (ii) violaxanthin (7.36 min); (iii) capsanthin (8.89 min); (iv) cis-capsanthin (10.33); (v) capsolutein (10.83 min); (vi) Zeaxanthin (11.2 min); (vii) cis-Zeaxanthin (12.0 min); (viii) β-crypotxanthin (14.36 min); and (ix) β-carotene. The disadvantages of chemical saponification are discussed hereinabove.

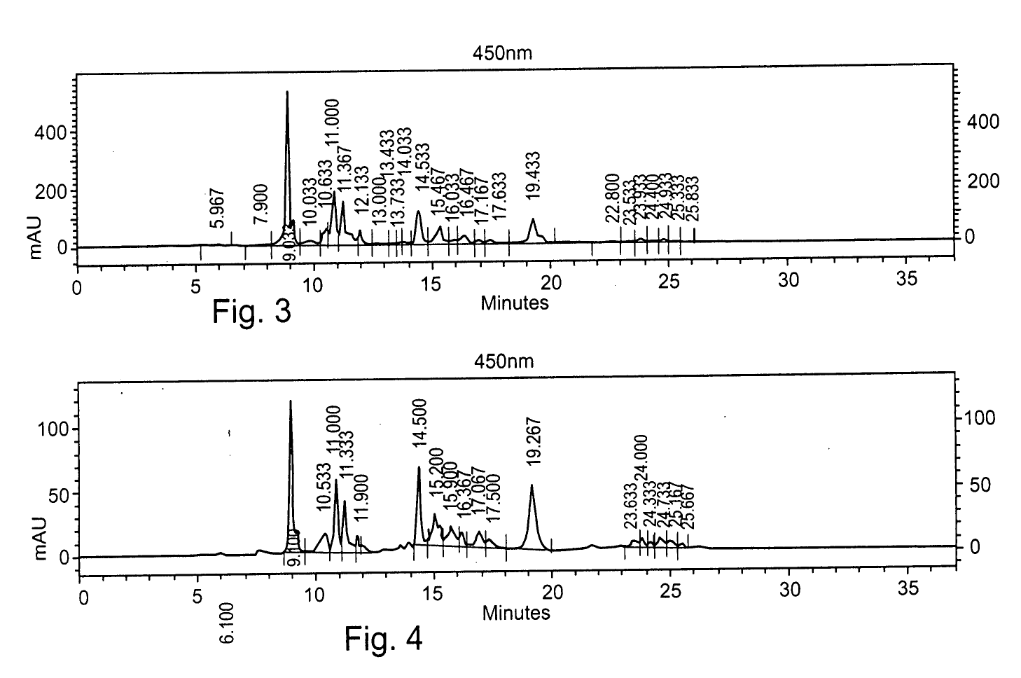

FIG. 3 demonstrates that incubation of red pepper (paprika) at 37 C for 24 hours with a pectinase/cellulase (Rohament max (Rohm) 0.1% by weight), a protease (Corolase PN-L (Rohm) 0.1% by weight) that macerate the pectins, proteins and cellulose, respectively, and a lipase (amano 30, 0.1% by weight), results in deesterification of the monoesters and diesters to the free carotenoids yielding a chromatogram which is similar to the chromatogram obtained via chemical deesterification (FIG. 2).

FIG. 4 demonstrates deesterification of paprika oleoresin following incubation of the oleoresin in the presence of deoxycholate (4% by weight) and lipase (amano 30, 0.1% by weight) for 24 hours at 37° C.

Similar assays conducted with other lipases: pancreatic lipase, lipase A “Amano 6”, lipase F-AP 15 gave far poorer results.

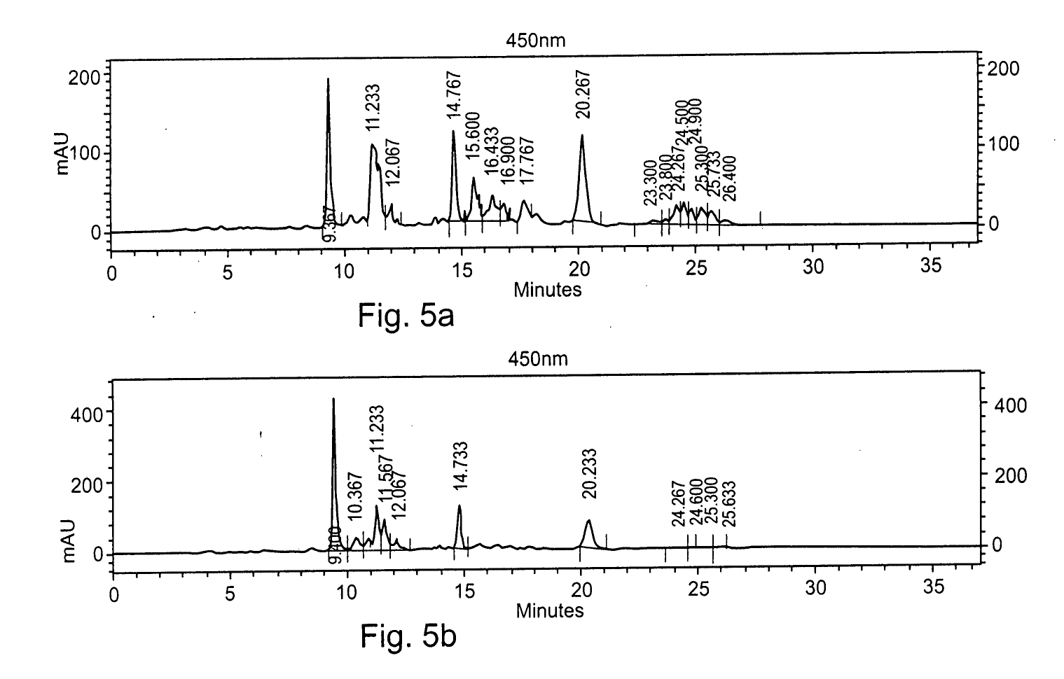

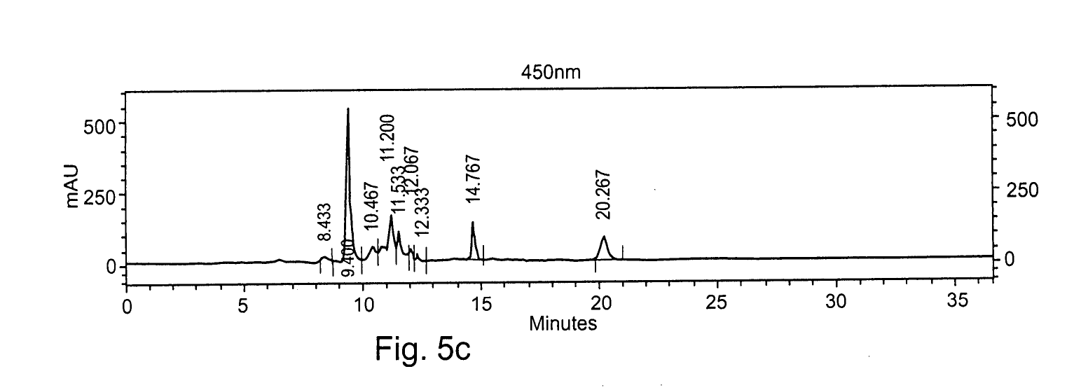

FIGS. 5a-c demonstrate deesterification of paprika oleoresin following incubation of the oleoresin in the presence of deoxycholate (2%, 3% or 4% by weight, respectively) and lipase (amano 30, 0.1% by weight) for 48 hours at 37° C. Note that similar carotenoid deesterification results are obtained with 3% and 4% deoxycholate, yet somewhat inferior carotenoid deesterification results are obtained with 2% deoxycholate. It will be appreciated that similar reaction optimizations can be performed for all other reaction ingredients.

These results demonstrate that it is possible to efficiently deesterify red pepper carotenoids by esterases. Enzymatic deesterification of the paprika carotenoids, prior to ingestion by human or animals enhances very much the bioavailability of these compound from the gut to the plasma.

### EXAMPLE 3

### The Effect of CaCl₂ and NaCl to the Lipase Activity

The activity of lipase at pH 7.6 at 37.0° C. for 18 hours on the deestrification of red-pepper carotenoids was measured in the presence of CaCl₂ and NaCl. As shown in Table 1, below, the addition of CaCl₂ to the reaction mixture, significantly increased lipase activity.

TABLE 1

| Treatment | % Deestrification |
|---|---|
| Enzyme alone* | 73 |
| Enzyme + CaCl₂ 1.875 mM | 78 |
| Enzyme + CaCl₂ 3.75 mM | 82 |
| Enzyme + CaCl₂ 7.5 mM | 89 |

*50 mg oleoresin, 400 mg deoxycholate, 250 mg lipase.

In the presence of 150 mM NaCl without CaCl₂, the deestrification was of 87%.

### EXAMPLE 4

### Extraction of Oleoresin From Fresh or Frozen Red-Pepper Fruits

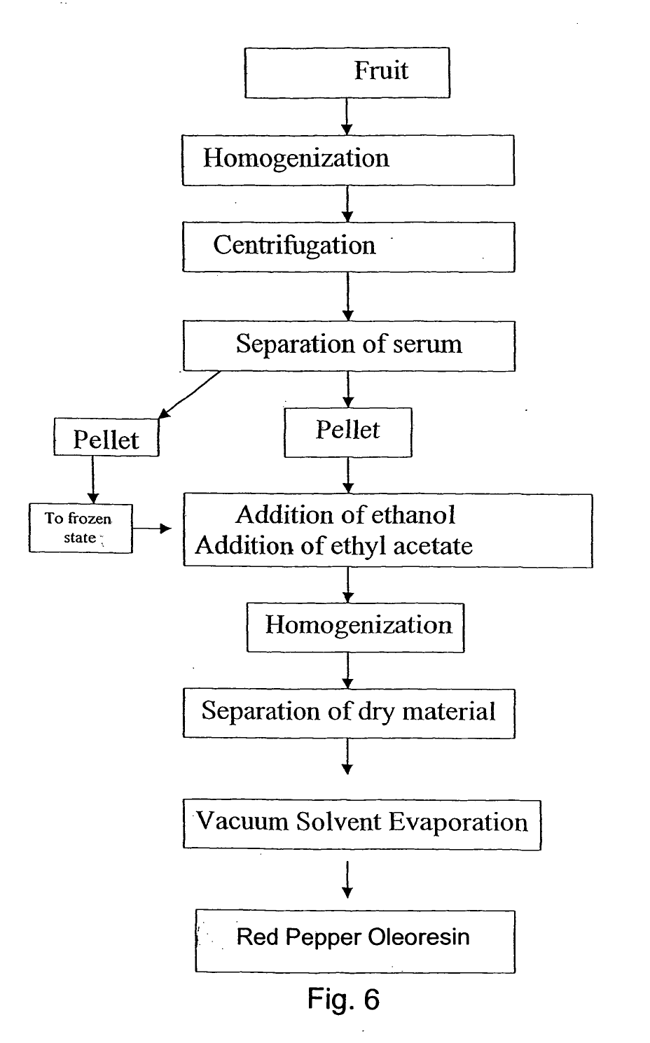

Fresh or frozen red-pepper fruits (100 parts) were homogenized with distilled water (40 parts) for 5 minutes to a juice. The juice was centrifuged at 25,000 g for 20 minutes and the pellet, either directly, or frozen, was mixed with 2 parts of ethanol and 10 parts of ethyl acetate. The elluent was homogenized for 1 minute. The solvents were separated from the dry material by centrifugation and evaporated at 45° C. under vacuum to receive red pepper oleoresin. The steps of the method are schematically presented in the flow chart of FIG. 6.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED

### (Additional References are Cited in the Text)

Akhtar P., Gray I J., Thomas H C., Garling D L. And Booren Am. Dietary pigmentation and carotenoids in rainbow trout muscle and liver tissue. J. Food Chem. 1999, 64, 234-239.

Aviram M. Review of human studies on oxidative damage and antioxidant protection related to cardiovascular diseases. Free Radic. Res. 1999, (in press).

Aviram M. Paraoxonase protects lipoproteins foam oxidation and attenuates atherosclerosis. Cardiovas. Res. 1999 (in press).

Aviram M, Maro I, Keidar S, Hayck T et al., Lesioned low-density lipoprotein in atheroscelrotic aplipoprotein E-deficient transgenic mice and human is oxidized and aggregated. Biochem. Biophys. Res. Commun. 1995, 16, 501-513.

Aviram M. Oxidized low density lipoproteins (OX-LDL) interaction with macrophages in atherosclerosis and the antiatherogenicity of antioxidants. Europ. J. Clin. Chem. Clin Biochem. 1996, 34, 599-608.

Birchall M A, Schock E, Harmon B V, Gobe G. Apoptosis, mitosis, PCNA and bcl-2 in normal, leukoplakic and malignant epithelia of the human oral cavity: prospective, in vivo study. Oral Oncol 1997,33, 419-425

Block G, Patterson B, Subar A. Fruit, vegetables and cancer prevention. A review of the epidemiological evidance. Nutr. Cancer, 1992, 18, 3-4.

Bohm F, Edge R, Land J E, McGravey DJ, Triscott J G. Carotenoids enhance vitamin E antioxidant efficiency. J. Am. Chem. Soc. 1997, 119, 621-622.

Bosland P W. Breeding for quality in Capsicum. Capsicum Eggplan Newsl. 1993, 12, 25-28.

Bras A, Sanches R, Cristovao L, et al. Oxidative stress in familial adenomatous polyposis. Eur J Cancer Prev 1999, 8, 305-310.

Britton G. In Natural Food Clorants (Hendry G A Fand Houghton J. D. eds) Blockie Academic Professional, London, 1996, p. 197.

Bundgaard T, Wildt J, Frydenberg M, Elbrond O, Nielsen J E. Case-control study of squamous cell cancer of the oral cavity in Denmark. Crit Rev Oral Biol Med 1995, 6, 5-17.

Burton G W, Ingold K U. β-carotene: An unusual type of lipid antioxidant. Science, 1984, 224, 569-573.

Collins A R, Gedik C M, Olmedilla B, Southon S, Bellizi M. Oxidative D NA damage measured in human lymphocytes: large differences between sexes and between countries, and correlation with mortality rates. FASEB J 1998, 12, 1397-400.

Cowan C G, Calwell E I L, Young I S, McKillop DJ, Lamey P-J: Antioxidant status of oral mucosal tissue and plasma levels in smokers and non-smokers. J Oral Path Med 1999, 28, 360-363.

Dammer R, Neiderdellman H, Friesenecker J, Fleisschmann H, Hermann J, Kreft M. Withdrawal therapy of patients with alcoholism and nicotine dependence with carcinomas in the area of the head a neck. Luxury or necessity? Carcinogenesis 1998, 19, 509-514.

De Stefani E, Boffetta P, Oreggia F, Mendilaharsu M, Deneo-Pellegrini H. Smoking patterns and cancer of the oral cavity and pharynx: a case control study in Urugay. Indian J Cancer 1998, 35, 65-72.

Dugas T R, Morel D W, Harrison E H. Dietary supplementation with β-carotene, but not with lycopene, inhibits endothelial all-mediated oxidation of low-density lipoprotein. Free Rad. Biol. Med. 1999, 26, 1238-1244.

Esterbauer H, Dieber-Rotheneder M, Striegl G, Waeg G. Role of vitamin E in preventing the oxidation of low-density lipoproteins. Am. J. Chim. Nutr. 1991, 53, 3145-3215.

Esterbaur H, Cheseman K H. Determination of aldehydic lypid peroxidation products: malonaldehyde and 4-hydroxynonenal. Methods Enzymol 186, 407-421.

Everett J A, Dennis M F, Patel K B, Maddix S, Kunder S C, Wilson R L. Scavenging of nitrogen dioxide, thiyl and sulfonyl free radicals by the nutritional antioxidant β-carotene. J. Biol. Chem. 1996, 271, 2988-2991.

Frankel E N, Kanner J. German J B, Kinsella E J. Inhibition of oxidation of human low-density lipoprotein with phenolic substances in red-wine Lancet 1993, 341, 454-457.

Fuhrman B, Elis A. and Aviram M. Antiathrogenic effects of lycopene and β-carotene: inhibition of LDL oxidation, and suppression of cellular cholesterol synthesis. Natural Antioxidants and Anticarcinogenesis in Nutrition Health and Disease. Eds. Kumpulainen J. T. and Salonen J. T. Society of Chemistry, Cambridge, U.K. 1999, pp. 226-230.

Fuhrman B, Lavy A, and Aviram M. Consumption of red wine with meals reduces the susceptibility of human plasma and LDL to undergo lipid peroxidation. Am. J. Clin. Nutr. 1995, 61, 549-554.

Gaziano J M, Hatta A, Flynn M, Johnson E J et al., NI, Ridker P M, Henekens C H, Frei B. Supplementation with beta-carotene in vivo and in vitro does not inhibit low density lipoprotein oxidation. Atherosclerosis 1995, 112, 187-195.

Gerster H. The potential role of lycopene for human health. J. Am. Cell. Nutr. 1997, 16, 109-126.

Goldsworthy T L, Conolly R B, Fransson-Steen R. Apoptosis and cancer risk assessment. Mutat Res 1996, 365, 71-90.

Goodwin T W: “The Biochemistry of the Carotenoids” Vol. 1: “Plants”. New York, Chapman and Hall, 1980, p. 203.

Gravis G, Pech-Gourgh F, Viens P, Alzieu C, Camerlo J, Oziel-Taieb S, Jausseran M, Maraninchi D. Phase II study of a combination of low-dose 13-cis-retinoic acid and interferon-alpha in patients with advanced head and neck squamous cell carcinoma. Anticancer Drugs 1999, 10, 369-374.

Halliwell B. Cellular stress and protection mechanism. Biochem. Soc. Trans. 1996, 24, 1023-1027.

Hart Ak, Karakala D W, Pitman K T, Adams J F. Oral and oropharyngeal squamous cell carcinoma in young adults: a report on 13 cases and review of the literature. Carcinogenesis 1999, 20 743-748.

Hennekens C H, Buring J E, Manson J E, Stampfer M et al. Lack of effect of long-term supplementation with beta-carotene on the incidence of malignant neoplams and cardiovascular disease. N. Engl. J. Med. 1996, 334, 1145-1149.

Hertog M G L, Feskens E J M, Hollman P C H, Katan M B, et al. Dietary antioxidants flavonoids and risk of coronary heart disease: The Zutphen Eldery Study Lancet 1993, 342, 1007-1011.

Hirayama O, Nakamura K, Hamda S, Kobayasi Y. Singlet oxygen quenching ability of naturally occurring carotenoids. Lipid, 1994, 29, 149-151.

Hong W K, Lippman S M, Itri L M, et al. Prevention of second primary tumors with isotretinoin in squamous-cell carcinoma of the head and neck. N Engl J Med 1990; 323:795-801

Ilyas M, Straub J, Tomlinson I P, BodmerWF. Genetic pathways in colorectal and other cancers. Eur J Cancer 1999, 35, 335-351.

Iribarren C, Folsom A R, Jacobs D R Jr et al. Association of serum vitamin levels, LDL susceptibility to oxidation and autoantibodies against MDA-LDL with carotid atherosclerosis. Arterioscler. Tromb. Vase Biol. 1997, 17, 1171-1177.

Jain C K, Agarval S, Venketeshwer R. The effect of dietary lycopene on bioavailability, tissue distribution, in vivo antioxidant properties and colonic preneoplasia in rats. Nutr Res 1999, 191, 383-391.

Kanner J, and Kinsella, J E, Lipid deterioration: β-carotene destruction and oxygen evolution in a system containing lactoperoxidase, hydrogen peroxide and halides. Lipids. 1983, 18, 198.

Kanner J, Frankel E, Granit R, German B, and Kinsella E, Natural antioxidants in grapes and wines. J. Agric. Food Chem. 1994, 42, 64-69.

Kennedy T A, Liebler D C. Peroxyl radical scavenging by β-carotene in lipid bilayers. J. Biol. Chem. 1992, 267, 4658-4663.

Khachik F. Beecher G R, Smith J C. Lutein, lycopene and their oxidative metabolites in chemoprevention of cancer. J. Cell Biochem. 1995, 22, 236-246.

Kim D J, Takasuka N, Kim J M, Sekine K, Ota T, Asamoto M, Murakoshi M, Nishino H, Nir Z, Tsuda H (1997) Chemoprevention by lycopene of mouse lung neoplasia after combined initiation treatment with DEN, MNU and DMH. Cancer Lett 120,15-22.

Kim J M, Araki S, Kim D J, Park C B, Takasuka N, Baba-Toriyama H, Ota T, Nir Z, Khachik F, Shimidzu N, Tanaka Y, Osawa T, Uraji T, Murakoshi M, Nishino H, Tsuda H (1998) Chemopreventive effects of carotenoids and curcumins on mouse colon carcinogenesis after 1,2-dimethylhydrazine initiation. Carcinogenesis 19,81-85.

Knekt P, Jarvinen R, Reunaneu A, Maatek. Flavonoid intake and coronary mortality in Finland: a cohort study. Brit. Med. J. 1996, 312, 478-481.

Knudsen K E, Weber E, Arden K C, Cavenee W K, Feramisco J R, Knudsen E S. The retinoblastoma tumor suppressor inhibits cellular proliferation through two distinct mechanisms inhibition of cell cycle progression and induction of cell death. Oncogene 1999, 16, 5239-5245.

Kohimeier L, Hossting S B. Epidemiologic evidence of a role of carotenoids in cardiovascular disese prevention. Am. J. Clin. Nutr. 1995, 62, 137s⁻¹⁴⁶s.

Kohlmeier L, Kark J D, Gomez-Grania E, et al. Lycopene and mycoradial infraction risk in the EURAMIC study. Am. J. Epidemiol. 1997, 146, 618-622.

Kondo K, Matsumoto Ak, Kusata H, Tenahashi H, Koda H, et al. Inhibition of oxidation of low-density lipoprotein with red-wine. Lancet, 344, 1152-1152.

Kristenson M, Zieden B, Kuinkiene S, et al. Antioxidant state and mortality from coronary heart disease in Lithuanian and Swedish men. B. M. J. 1997, 314, 629-632.

Lapidot, T. Harel, S. Akiri, B. Granit, R. and Kanner, J. PH-Dependent forms of red wine anthocyanins as antioxidants. J. Agric. Food Chem. 1999, 47, 67-70.

Lapidot, T. Harel, S. Granit, R. Kanner, J. Anthocyanins in red wines: Antioxidant activity and bioavailability in human. In Natural 1999, 151-161.

Lee C M. Borleau A. Boileau T W M, Williams A W. Et al. Review of animal models in carotenoid research. J. Nutr. 1999, 129, 2271-2277.

Lee I M. Cook N R. Monson J E. Buring J E. Hennekens C H. β-carotene supplementation and incidence of cancer and cardiovascular disease: the women's study. J. Natl. Cancer Inst. 1999, 91, 2102-2102.

Lefebvre V, Kunz M, Camara B. and Palloix A. The capsanthin-capsorubin synthase gene: candidate for the y locus controlling the red fruit color in pepper. Plant Molec. Biol. 1998. 36, 785-789.

Levy A, Harel S, Palevich D, Akiri B, Menagem E, and Kanner J. Carotenoid pigments and β-carotene in paprika fruit (Capsicum spp.) with different genotypes. J. Agric. Food Chem. 1995, 43, 362-367.

Levy A, Levy Talia, S, Elikin Y, Menagem E, Barzilai M, and Kanner J. Carotenoid and vitamin C and E contents in isogenic chlorophyll and color mutants of paprika (Capsicum annuum L.). Proc. Xth. Eucarpia Meeting on Genetics and Breeding of Capsicum and Eggplant. 1998, 257-260.

Levy J. Bosin E, Feldman B, Giat Y et al. Lycopene is a more potent inhibitor of human cancer cell proliferation than lither α-carotene of β-carotene. Nutr. Cancer 1995, 24, 257-267.

Lin Y, Burri B J, Neidlinger T R, Muller H G, Ducker S R, Cliford A J. Estimating the concentration of beta-carotene required for maximal protection of low-density lipoprotein in women. Am. J. Clin. Nutr. 1998, 67, 837-845.

Mao L. Leukoplakia: Molecular understanding of pre-malignant lesions and implications for clinical management. Mol Med Today 1997, 3, 442-448

Mathews Roth M M, Welankiwar S, Sehgal P K, Lausen N L G et al. Distribution of (¹⁴C) lycopene in rats and monkey. J. Nutr. 1990, 120, 1205-1213.

Matsufuji H, Nakamura H, Chino M and Takeda M. Antioxidant activity of capsanthin and the fatty acid estess in paprika (Capsicum annuum). J. Agric. Food Chem. 1998, 46-49.

Mayne S T, Beta-carotene, carotenoids and disease prevention in human, FASEB J. 1996, 10, 690-699.

Murakoshi M, Nishino H, Satomi Y, Takayasu J et al. Potent preventive action of α-carotene against carcinogenesis spontaneous liver carcinogenesis in mice are suppressed more effectively by β-carotene. Cancer Res. 1992, 52, 6583-6587.

Narisawa T, Fukaura Y, Hasebe M, Ito M, Aizawa R, Murakoshi M, Uemura S, Khachik F, Nishino H (1996) Inhibitory effects of natural carotenoids, alpha-carotene, beta-carotene, lycopene and lutein, on colonic aberrant crypt foci formation in rats. Cancer Lett 107,137-142.

Ojima F, Sakamoto H, Ishiguro Y, Ferao J. Consumption of carotenoids in photosensitized oxidation of human plasma and low-density lipoprotein. Free Rad. Biol. Med. 1993, 15, 377-384.

Olson J A. Carotenoids, In: Modem Nutrition in Health and Disease (Shils M E, Olson J A, Shike M. & Ross A C eds) Williams and Wilkins, Baltimore, Md. 1999, p. 525.

Oshima S, Sakamoto H, Ishiguro Y and Terao J. Accumulation and cleamce of capsanthin in blood plasma after the ingestion of paprika juice in men. J. Nut. 1997, 127, 1475-1479.

Pappalardo G, Maiani G, Mobarhan S, Guadalaxara A, Azzini E, Raguzzini A, Salucci M, Serafini M, Trifero M, Illomei G, Ferro-Luzzi A (1997) Plasma (carotenoids, retinol, alpha-tocopherol) and tissue (carotenoids) levels after supplementation with beta-carotene in subjects with precancerous and cancerous lesions of sigmoid colon. Eur J Clin Nutr 51, 661-666.

Poulos J. Pepper breeding (Capsicum spp.): achievements, challenges and possibilities. Plant Breeding Absr. 1994, 64, 143-146.

Rao A V and Aganval S. Role of lycopene as antioxidant carotenoid in the prevention of chronic disease: A review. Nutr. Res. 1999, 19, 305-323.

Reaven P D, Ferguson E, Navab M, Powell F L. Susceptibility of human LDL to oxidative modification. Effects of variations in beta-carotene concentration and oxygen tension. Alterioscler. Troub. 1994, 14, 1162-1169.

Romanchik J E, Morel D W, Horrison E H. Distribution of carotenoids and alpha-tocopherol among lipoproteins do not change when human plasma is incubated in vitro. J. Nutr. 1995, 88, 1646-1650.

Ross R. The pathogenesis of atherosclerosis: a perspective for the 1990. Nature, 1993, 362, 801-809.

Schildt E B, Eriksson M, Hardell M, Magnuson A. Oral snuff, smoking habits and alcohol consumption in relation to oral cancer in a Swedish case-control study. Int J Cancer 1998, 77, 333-336

Schoelch M L, Le Q T, Silverman S Jr, McMillan A, Dekker N P, Fu K K, Ziober B L, Regezi J A. Apoptosis-associated proteins and the development of oral squamous cell carcinoma. Oral Oncol 1999, 35, 77-85.

Schroeder W A and Johnson E A. Singlet oxygen and peroxyl radical regulate carotenoid biosynthesis in Phaffia Rhodozyma. J. Biol. Chem. 1995, 270, 18374-18379.

Schwartz J L and Shklar G. Retinoid and carotenoid angiogenesis: a possible explanation for enhanced oral carcinogenesis. Nutr Cancer 1997, 27, 192-99.

Schwartz J L and Shklar G. The selective cytotoxic effect of carotenoids and alpha tocopherol on human cancer cell lines in vitro. J Oral Maxill Surg 1992, 50, 367-373.

Schwartz J L Tanaka J, Khandekar V, Herman T S, Teicher B. Beta carotene and/or vitamin E as modulators of alkakylating agents in SSC-25 human squamous carcinoma cells. Cancer Chem and Pharmacol 1991, 29, 207-213.

Schwartz J L, Antoniades D Z, Zhao S. Molecular and biochemical reprogramming of oncogenesis through the activity of antioxidants and prooxidants. Ann NY Acad Sci 1992, 686, 292-279.

Schwartz J L, Flynn E A, Shklar G. The effect of carotenoids on antitumor immune response in vivo and in vitro with hamster and mouse immune effectors. Ann NY Acad Sci 1990, 587, 92-109.

Schwartz J L, Shklar G, Trickler D. p53 in the anticancer mechanism of vitamin E. Oral Oncol 1993, 29B, 313-183.

Sies H, Stahl W. Vitamins E, C, β-carotene and other carotenoids as antioxidants as antioxidants. Am. J. Clin. Nutr. 1995, 62, 1315-1321.

Smith E M, Hoffman H T, Summersgill K S, Kirchner H L, Turek L P, Haugen T H. Human papillomavirus and risk of oral cancer. Int J Cancer 1998, 77, 341-346

Stahl W, Junghans A, deBoer B, Driomina E S. et al. Caroteoid mixtures protect multieamillar liposomes against oxidative damage; synergistic effects of lycopene and lutein. FEBS Lett 1998, 427, 305-308.

Steinberg D, et al. Antioxidants in the prevention of human atheroscelrosis. Summary of the proceedings of a National Heart, Lung and Blood Institute Workshop: Circulation 1992, 85, 2337-2344.

Steinberg D, Parthasarathy S, Carew T E, Khoo J C and Witztum J L. Beyond cholesterol: modifications of low-density lipoprotein that increase its atherogenecity. N Engl. J. Med. 1989, 320, 915-924.

Sthal W, Sies H. Uptake of lycopene and its geometrical isomers is greater from heat-processed than form unprocessed tomato juice in humans. J. Nutr. 1992, 122, 2161-2166.

Stich H F, Roisin M P, Hornby A P et al: Remission of oral leukoplakias and micronuclei in tobacco/betel quid chewers treated with beta-carotene and with beta-carotene plus vitamin A. Int J Cancer 1998, 421, 195-199.

Tanaka T, Morishita Y, Suzui M, Kojima T et al. Chemo prevention of mouse urinary bladder carcinogenesis by the naturally occuring carotenoid astaxanthin. Carcinogenesis. 1994, 15, 15-19.

Wagner J R, Motchnik P A, Stocker R, Sies H, Ames B N. The oxidation of blood plasma and low-density lipoprotein components by chemically generated single oxygen. J. Biol. Chem. 1993, 268, 18502-18506.

Watson A D, Navab M, Hama S Y, Sevanian A et al. Effect of platelet activating factor-acetyl hydrolase on the formation and action of minimally oxidized low-density lipoproteins. J. Clin. Invest. 1995, 95, 774-782.

Weisburger J H. Mechanisms of action of antioxidants as exemplified in vegetables, tomatoes and tea. Food Chem Toxicol 1999, 37, 943-948.

Woodall A A, Lee S W, Wesie R J, Jackson M J and Britton G. Oxidation of carotenoids by free radicals: relationship between structure and reactivity. Biochim. Biophys. Acta 1997, 1336, 33-42.

Yao I, Iwai M, Furuta I. Correlation of bcl-2 and p53 expression with clinicopathological features in tongue sqamous cell carcinomas. Oral Oncol 1999, 35, 56-62.

Yla-Herttuala S, Palinski W, Rosenfeld M E, Parthasarathy S. et al. Evidance for the presence of oxidatively modified low-density lipoprotein in atherosclerotic lesions of rabbit and mice. J. Clin. Invest. 1989, 84, 1086-1095.

Ziegler R G, A view of the epidemiological evidance that carotenoids reduce the risk of cancer. J. Nutr. 1988, 119, 116-122.

What is claimed is:

1. A method of determining an efficiency of an esterase in increasing a fraction of free carotenoids in a source of carotenoids in which at least some of the carotenoids are fatty acid esterified carotenoids, the method comprising:
contacting the source of carotenoids with the esterase under preselected experimental conditions; and
using a carotenoids detection assay for determining the efficiency of the esterase in increasing the fraction of the free carotenoids in the source of carotenoids.

2. The method of claim 1, wherein said source of carotenoids is characterized in that a majority of the carotenoids in said source of carotenoids are said fatty acid esterified carotenoids.

3. The method of claim 1, wherein said source of carotenoids is red pepper.

4. The method of claim 1, wherein said source of carotenoids is red pepper powder.

5. The method of claim 1, wherein said source of carotenoids is paprika.

6. The method of claim 1, wherein said source of carotenoids is red pepper oil extract.

7. The method of claim 1, wherein said source of carotenoids is red pepper oleoresin.

8. The method of claim 1, wherein said source of carotenoids is selected from the group consisting of apple, apricot, avocado, blood orange cape goosberry, carambola, chilli, clementine, kumquat, loquat, mango, minneola, nactarine, orange, papaya, peach, persimmon, plum, potato, pumpkin, tangerine and zucchini.

9. The method of claim 1, wherein said esterase is selected from the group consisting of a lipase, a carboxyl ester esterase and a chlorophylase.

10. The method of claim 1, wherein said esterase is a lipase.

11. The method of claim 10, wherein said lipase is selected from the group consisting of bacterial lipase, yeast lipase, mold lipase and animal lipase.

12. The method of claim 1, wherein said esterase is immobilized.

13. The method of claim 1, wherein said preselected experimental conditions comprise at least one of:
addition of a cellulose degrading enzyme;
addition of a proteins degrading enzyme;
addition of a pectin degrading enzyme; and-
addition of an emulsifier.

14. The method of claim 13, wherein said cellulose degrading enzyme is selected from the group consisting of C₁ type beta-1,4 glucanase, exo-beta-1,4 glucanase, endo-beta-1,4 glucanase and beta-glucosidase.

15. The method of claim 13, wherein said proteins degrading enzyme is selected from the group consisting of tripsin, papain, chymotripsins, ficin, bromelin, cathepsins and rennin.

16. The method of claim 13, wherein said pectin degrading enzyme is selected from the group consisting of a pectinestrerase, pectate lyase and a polygalacturonase.

17. The method of claim 13, wherein said emulsifier is a non-ester emulsifier.

18. The method of claim 17, wherein said emulsifier is lecithin.

19. The method of claim 17, wherein said emulsifier is deoxycholate.

20. The method of claim 17, wherein said emulsifier is a non-ionic detergent.

21. The method of claim 17, wherein said emulsifier is derived from bile, gum Arabic or salt of free fatty acids.

22. The method of claim 1, wherein said carotenoids detection assay is a chromatography assay.

23. The method of claim 22, wherein said chromatography assay is selected from the group consisting of thin layer chromatography and high performance liquid chromatography.

24. A method of screening for esterases efficient in increasing a fraction of free carotenoids in a source of carotenoids in which at least some of the carotenoids are fatty acid esterified carotenoids, the method comprising:
contacting the source of carotenoids separately with each of the esterases under preselected experimental conditions; and
using a carotenoids detection assay for determining the efficiency of each of the esterases in increasing the fraction of the free carotenoids in the source of carotenoids, thereby screening for esterases efficient in increasing the fraction of free carotenoids in the source of carotenoids.

25. The method of claim 24, wherein said source of carotenoids is characterized in that a majority of the carotenoids in said source of carotenoids are said fatty acid esterified carotenoids.

26. The method of claim 24, wherein said source of carotenoids is red pepper.

27. The method of claim 24, wherein said source of carotenoids is red pepper powder.

28. The method of claim 24, wherein said source of carotenoids is paprika.

29. The method of claim 24, wherein said source of carotenoids is red pepper oil extract.

30. The method of claim 24, wherein said source of carotenoids is red pepper oleoresin.

31. The method of claim 24, wherein said source of carotenoids is selected from the group consisting of apple, apricot, avocado, blood orange cape goosberry, carambola, chilli, clementine, kumquat, loquat, mango, minneola, nactarine, orange, papaya, peach, persimmon, plum, potato, pumpkin, tangerine and zucchini.

32. The method of claim 24, wherein said esterases are selected from the group consisting of lipases, carboxyl ester esterases and chlorophylases.

33. The method of claim 24, wherein said esterases are lipases.

34. The method of claim 33, wherein said lipases are selected from the group consisting of bacterial lipases, yeast lipases, mold lipases and animal lipases.

35. The method of claim 24, wherein said esterases are immobilized.

36. The method of claim 24, wherein said preselected experimental conditions comprise at least one of:
addition of a cellulose degrading enzyme;
addition of a proteins degrading enzyme;
addition of a pectin degrading enzyme; and
addition of an emulsifier.

37. The method of claim 36, wherein said cellulose degrading enzyme is selected from the group consisting of Cl type beta-1,4 glucanase, exo-beta-1,4 glucanase, endo-beta-1,4 glucanase and beta-glucosidase.

38. The method of claim 36, wherein said proteins degrading enzyme is selected from the group consisting of tripsin, papain, chymotripsins, ficin, bromelin, cathepsins and rennin.

39. The method of claim 36, wherein said pectin degrading enzyme is selected from the group consisting of a pectinestrerase, pectate lyase and a polygalacturonase.

40. The method of claim 36, wherein said emulsifier is a non-ester emulsifier.

41. The method of claim 40, wherein said emulsifier is lecithin.

42. The method of claim 40, wherein said emulsifier is deoxycholate.

43. The method of claim 40, wherein said emulsifier is a non-ionic detergent.

44. The method of claim 40, wherein said emulsifier is derived from bile, gum Arabic or salt of free fatty acids.

45. The method of claim 24, wherein said carotenoids detection assay is a chromatography assay.

46. The method of claim 45, wherein said chromatography assay is selected from the group consisting of thin layer chromatography and high performance liquid chromatography.

47. A method of optimizing reaction conditions for increasing a fraction of free carotenoids in a source of carotenoids in which at least some of the carotenoids are fatty acid esterified carotenoids, via an esterase, the method comprising:
contacting the source of carotenoids with the esterase under different preselected experimental conditions; and
using a carotenoids detection assay for determining the efficiency of the esterase in increasing the fraction of the free carotenoids in the source of carotenoids under each of said different preselected experimental conditions, thereby optimizing the reaction conditions for increasing the fraction of free carotenoids in the source of carotenoids in which at least some of the carotenoids are fatty acid esterified carotenoids via the esterase.

48. The method of claim 47, wherein said source of carotenoids is characterized in that a majority of the carotenoids in said source of carotenoids are said fatty acid esterified carotenoids.

49. The method of claim 47, wherein said source of carotenoids is red pepper.

50. The method of claim 47, wherein said source of carotenoids is red pepper powder.

51. The method of claim 47, wherein said source of carotenoids is paprika.

52. The method of claim 47, wherein said source of carotenoids is red pepper oil extract.

53. The method of claim 47, wherein said source of carotenoids is red pepper oleoresin.

54. The method of claim 47, wherein said source of carotenoids is selected from the group consisting of apple, apricot, avocado, blood orange cape goosberry, carambola, chilli, clementine, kumquat, loquat, mango, minneola, nactarine, orange, papaya, peach, persimmon, plum, potato, pumpkin, tangerine and zucchini.

55. The method of claim 47, wherein said esterase is selected from the group consisting of a lipase, a carboxyl ester esterase and a chlorophylase.

56. The method of claim 47, wherein said esterase is a lipase.

57. The method of claim 56, wherein said lipase is selected from the group consisting of bacterial lipase, yeast lipase, mold lipase and animal lipase.

58. The method of claim 47, wherein said esterase is immobilized.

59. The method of claim 47, wherein said different preselected experimental conditions comprise at least one of:
addition of a cellulose degrading enzyme;
addition of a proteins degrading enzyme;
addition of a pectin degrading enzyme; and
addition of an emulsifier.

60. The method of claim 59, wherein said cellulose degrading enzyme is selected from the group consisting of C 1 type beta-1,4 glucanase, exo-beta-1,4 glucanase, endo-beta-1,4 glucanase and beta-glucosidase.

61. The method of claim 59, wherein said proteins degrading enzyme is selected from the group consisting of tripsin, papain, chymotripsins, ficin, bromelin, cathepsins and rennin.

62. The method of claim 59, wherein said pectin degrading enzyme is selected from the group consisting of a pectinestrerase, pectate lyase and a polygalacturonase.

63. The method of claim 59, wherein said emulsifier is a non-ester emulsifier.

64. The method of claim 63, wherein said emulsifier is lecithin.

65. The method of claim 63, wherein said emulsifier is deoxycholate.

66. The method of claim 63, wherein said emulsifier is a non-ionic detergent.

67. The method of claim 63, wherein said emulsifier is derived from bile, gum Arabic or salt of free fatty acids.

68. The method of claim 47, wherein said carotenoids detection assay is a chromatography assay.

69. The method of claim 68, wherein said chromatography assay is selected from the group consisting of thin layer chromatography and high performance liquid chromatography.

70. A method of increasing a fraction of free carotenoids in a source of carotenoids in which at least some of the carotenoids are fatty acid esterified carotenoids, the method comprising contacting the source of carotenoids with an effective amount of an esterase under conditions effective in deesterifying the fatty acid esterified carotenoids, thereby increasing the fraction of free carotenoids in the source of carotenoids.

71. The method of claim 70, wherein said source of carotenoids is characterized in that a majority of the carotenoids in said source of carotenoids are said fatty acid esterified carotenoids.

72. The method of claim 70, wherein said source of carotenoids is red pepper.

73. The method of claim 70, wherein said source of carotenoids is red pepper powder.

74. The method of claim 70, wherein said source of carotenoids is paprika.

75. The method of claim 70, wherein said source of carotenoids is red pepper oil extract.

76. The method of claim 70, wherein said source of carotenoids is red pepper oleoresin.

77. The method of claim 70, wherein said source of carotenoids is selected from the group consisting of apple, apricot, avocado, blood orange cape goosberry, carambola, chilli, clementine, kumquat, loquat, mango, minneola, nactarine, orange, papaya, peach, persimmon, plum, potato, pumpkin, tangerine and zucchini.

78. The method of claim 70, wherein said esterase is selected from the group consisting of a lipase, a carboxyl ester esterase and a chlorophylase.

79. The method of claim 70, wherein said esterase is a lipase.

80. The method of claim 79, wherein said lipase is selected from the group consisting of bacterial lipase, yeast lipase, mold lipase and animal lipase.

81. The method of claim 70, wherein said esterase is immobilized.

82. The method of claim 70, wherein said conditions effective in deesterifying the fatty acid esterified carotenoids comprise at least one of:
addition of a cellulose degrading enzyme;
addition of a proteins degrading enzyme;
addition of a pectin degrading enzyme;
addition of an emulsifier; and
addition of at least one metal ion.

83. The method of claim 82, wherein said at least one metal ion is selected from the group consisting of Ca⁺⁺ and Na⁺.

84. The method of claim 82, wherein said addition of said at least one metal ion is by addition of at least one salt of said metal ion.

85. The method of claim 82, wherein said at least one salt is selected from the group consisting of CaCl₂ and NaCl.

86. The method of claim 82, wherein said cellulose degrading enzyme is selected from the group consisting of C 1 type beta-1,4 glucanase, exo-beta-1,4 glucanase, endo-beta-1,4 glucanase and beta-glucosidase.

87. The method of claim 82, wherein said proteins degrading enzyme is selected from the group consisting of tripsin, papain, chymotripsins, ficin, bromelin, cathepsins and rennin.

88. The method of claim 82, wherein said pectin degrading enzyme is selected from the group consisting of a pectinestrerase, pectate lyase and a polygalacturonase.

89. The method of claim 82, wherein said emulsifier is a non-ester emulsifier.

90. The method of claim 89, wherein said emulsifier is lecithin.

91. The method of claim 89, wherein said emulsifier is deoxycholate.

92. The method of claim 89, wherein said emulsifier is a non-ionic detergent.

93. The method of claim 89, wherein said emulsifier is derived from bile, gum Arabic or salt of free fatty acids.

94. The method of claim 70, further comprising extracting free carotenoids from the source of carotenoids.

95. A source of carotenoids having an increased fraction of free carotenoids and produced by the method of claim 70.

96. A food additive comprising the source of carotenoids of claim 95.

97. A feed additive comprising the source of carotenoids of claim 95.

98. A method of extracting red pepper oleoresin, the method comprising:
homogenizing red-pepper fruits in water into a juice;
centrifuging the juice so as to obtain a pellet;
mixing the pellet with ethanol and ethyl acetate;
homogenizing the pellet with the ethanol and the ethyl acetate;
removing dry material; and
evaporating solvents so as to obtain red pepper oleoresin.

99. The method of claim 98, wherein a weight ratio between said red-pepper fruits and said water is 80-120 parts of fruit to 20-60 parts of water.

100. The method of claim 98, wherein said red-pepper fruits are frozen.

101. The method of claim 98, wherein said red-pepper fruits are fresh.

102. The method of claim 98, wherein said juice is centrifuged at 20,000-30,000 g for 10-30 minutes.

103. The method of claim 98, wherein said pellet is mixed with 1-3 parts of said ethanol and 5-15 parts of said ethyl acetate.

104. The method of claim 98, wherein said removing dry material is by centrifugation.

105. The method of claim 98, wherein said evaporating solvents is at 40-50° C.

106. The method of claim 98, wherein said evaporating solvents is at 40-50° C. and under vacuum.

107. The method of claim 98, wherein said evaporating solvents is under vacuum.
